(19) United States
(12) Patent Application Publication
(10) Pub. No.: US 2023/0241124 A1
(43) Pub. Date: Aug. 3, 2023

(54) ISOTONIC LIPID EMULSION

(71) Applicants: NUTRITION LIPID DEVELOPMENTS, Brussels (BE); THE TRUSTEES OF COLUMBIA UNIVERSITY IN THE CITY OF NEW YORK, New York, NY (US)

(72) Inventors: Yvon CARPENTIER, Rhode-Saint-Genèse (BE); Richard J. DECKELBAUM, Hastings on Hudson, NY (US); Chuchun CHANG, New York, NY (US)

(21) Appl. No.: 18/015,622

(22) PCT Filed: Jul. 13, 2021
(86) PCT No.: PCT/EP2021/069438

§ 371 Date: Jan. 11, 2023

Related U.S. Application Data
(60) Provisional application No. 63/051,535, filed on Jul. 14, 2020.

(51) Int. Cl. A61K 35/60; A61K 9/107; A61K 31/202; A61K 31/685; A01N 1/02; A61K 45/06

(52) U.S. Cl. CPC A61K 35/60; A61K 9/107; A61K 31/202; A61K 31/685; A01N 1/0226; A61K 9/1075; A61K 45/06

(57) ABSTRACT

Isotonic lipid emulsion for use in the treatment of a number of diseases such as an acute heart ischemia, a brain ischemia or another organ damage which occurred on a day, where the treatment is applied by intravenous bolus injection which is repeated at time intervals during at least that day at which the disease occurred, the isotonic lipid emulsion comprising a 78 to 95% by weight of medium chain triglycerides and 5 to 22% by weight of a substance, said substance being either a fish oil, wherein the fish oil has an eicosapentaenoic acid content of from 20 to 50% by weight; or an eicosapentaenoic acid, or a docosapentaenoic acid, or a docosahexaenoic acid, or a combination of eicosapentaenoic acid, and/or docosapentaenoic acid, and/or docosahexaenoic acid.

The present invention relates to new uses and novel compositions of isotonic lipid emulsions in the treatment of a plurality of diseases such as, but not limited to, an acute heart ischemia, a brain ischemia or injury, a spinal cord injury, a severe surgical operation, an acute inflammatory reaction, septic and/or metabolic complications, a non-alcoholic fatty liver disease, an organ transplantation. The isotonic lipid emulsions according to the present invention can also be used in the treatment of skin or ocular diseases and for cosmetic applications on the skin. The used isotonic lipid emulsion comprises a 78 to 95% by weight of medium chain triglycerides and 5 to 22% by weight of a substance, said substance being either a fish oil, wherein the fish oil has an eicosapentaenoic acid content of from 20 to 50% by weight, and/or emulsions of compositions containing an eicosapentaenoic acid molecule, or a docosapentaenoic acid molecule, or a docosahexaenoic acid molecule, or a combination of the latter acid molecules, or esters and derivatives of these fatty acids, or microalgal oils.

Apart from the saturated and monounsaturated fatty acids (hereinafter abbreviated as FA), which are present in large amounts in current dietary intake, the polyunsaturated fatty acids have a substantial impact on health via their influence on cell membrane physical properties and on several biological reactions. These molecules are formed of two families, the omega-3 (or n-3) FA and the omega-6 (or n-6) FA series, depending on the position of their first unsaturation bond from the methyl (or omega) end of their chain. Each series consists of FA with different chain lengths—from 18 to 22 carbon atoms (C)—and degrees of unsaturation—from 2 to 6 unsaturated double bonds. They are collectively named essential fatty acids (hereinafter abbreviated as EFA's) since they cannot be synthetized by human beings. C18 precursors present in many vegetable oils, such as linoleate (C18:2 n-6) and α-linolenate (C18:3 n-3) in the n-6 and n-3 series, respectively are not biologically very active. However, these C18 precursors may be elongated and unsaturated, via a common elongases and desaturases enzyme systems. This implies a potential competition between both series to produce the longer and most biologically active fatty acids, i.e. the very long-chain fatty acids (hereinafter abbreviated as VLCFA): arachidonate (C20:4 n-6, hereinafter abbreviated as AA) in the n-6 series, and the group of eicosapentaenoate (C20:5 n-3, hereinafter abbreviated as EPA), docosapentaenoate (C22:5 n-3, hereinafter abbreviated as DPA), and docosohexaenoate (C22:6 n-3, hereinafter abbreviated as DHA) in the n-3 series. Thus, high dietary intake of C18:2 n-6 versus C18:3 n-3 will result into much higher concentrations of C20:4 n-6 versus reduced concentrations of C20:5 n-3 and C22:6 n-3 in plasma and in cell membranes of different organs of a human body.

These VLCFA are important constituents of phospholipids (hereinafter abbreviated as PL), the building blocks of outer and inner cell membranes and there is a competition between VLCFA from the n-6 and the n-3 series for being incorporated in cell membranes. Since most diets are richer in linoleate than in α-linolenate, the concentration of n-3 VLCFA in plasma and in cell membranes is generally quite lower than that of n-6 VLCFA, namely AA.

The presence in cell membranes of n-3 VLCFA, and namely of EPA, markedly increases membrane stability such as described by Mason R P, Libby P and Bhatt D L: Emerging mechanisms of cardiovascular protection for the omega-3 fatty acid eicosapentaenoic acid, Arteriosclerosis, Thrombosis and Vascular Biology, 2020, 40: 1135-1147—doi 10.1161. In the myocardium, high EPA content reduces cardiac excitability and lowers cardiac rate and protects against arrhythmias, presumably by augmenting vagal tone.

DHA is a major structural FA in particular for organs with their particular functions such as the retina and the brain. Higher concentrations of DHA in cell membranes increase membrane fluidity, which is particularly important in nervous cells, for ensuring proper function during synaptic and axonal growth. In addition, DHA has much greater effects on dissolving membrane lipid rafts than does EPA. Of interest, some cell receptors are localized in rafts.

Changes of n-3 VLCFA content in cell membranes affect signal transmission via ion channels and various receptors, and regulate the expression of several genes via actions on nuclear factors.

Apart from these direct effects, n-6 and n-3 VLCFA also exert important effects indirectly, via conversion into other active metabolites by enzymatic oxidation via the cyclooxygenase and lipoxygenase pathways, which leads to the production of eicosanoids: prostaglandins, leukotrienes, and thromboxanes. It has to be noted that mediators derived from AA generally tend to increase inflammatory and thrombotic reactions, while the n-3 derived counterparts have potent anti-inflammatory properties and anti-thrombotic activity, in particular by reducing platelet activation and adhesion as well as via thromboxane A2 synthesis and raising plasma concentration of plasminogen activator inhibitor-1. As well, omega-3 fatty acids, such as DHA, can act as antioxidants, inhibit apoptosis/necrosis, and preserve mitochondrial function after brain injury (Mayurasakorn K, Niatsetskaya Z V, Sosunov S A, Williams J J, Zirpoli H, Vlassakov I, Deckelbaum R J, and Ten V S, PLoS One 2011: e0160870. doi: 10.1371/journal.pone.0160870 2016). DHA, but not EPA emulsions preserve neurological and mitochondrial functions after brain hypoxia-ischemia in a neonatal mouse model of stroke).

Each of the three n-3 VLCFA is also the precursor for an array of specialized pro-resolving mediators (hereinafter abbreviated as SPM's) named protectins, resolvins and maresins which are selectively derived from EPA, DPA, and DHA (Serhan C N: Novel pro-resolving lipid mediators in inflammation are leads for resolution physiology, Nature, 2014; 510(7503): 92-101, doi: 10.1038/nature 13479). These SPM molecules not only exert very potent anti-inflammatory effects but do also facilitate resolution of the inflammatory process.

N-3 VLCFA molecules positively affect the endothelial function by raising nitric oxide (NO°) production, which results in arterial vasodilation, improved tissue micro-perfusion, lowering of blood pressure, and protection or restoration of arterial wall integrity.

Additional metabolic benefits of high n-3 FA intakes in acute conditions comprise: an increased sensitivity to insulin and improved glucose homeostasis, via a reduced ectopic fat deposition in muscles and a preserved muscular mass. High n-3 FA intake also lowers plasma triglyceride (hereinafter abbreviated as TG) concentrations in patients with hypertriglyceridemia.

Moreover, while very unsaturated FA are sensitive to peroxidative damage, increased content of n-3 VLCFA in cells and organs helps scavenging reactive oxygen species, protects against peroxidation, and maintains mitochondrial integrity and functionality in cells. EPA and DHA distinctly exert a substantial number of these beneficial properties, while the roles of DPA are still being elucidated.

Observational studies comparing populations with high versus. low consumption of marine fatty animals have reported better cardio-vascular health and a lower incidence of type 2 diabetes (T2D) and of some cancers in relation to high n-3 VLCFA intakes.

Unfortunately, the recent evolution of dietary habits in most regions of the world is characterized by a lower consumption of n-3 VLCFA together with a higher intake of n-6 FA.

After a period of enthusiasm for the prophylactic use of n-3 VLCFA in cardio-vascular disease prevention, many randomized control trials of fish oil (hereinafter abbreviated as FO) supplementation have led to disappointing results. This may be due to several factors such as an inadequate proportion of the different n-3 VLCFA, but also to the low FO quality of some dietary supplements (Sherratt S R S, Lero M, Mason R P: Are dietary fish oil supplements appropriate for dyslipidemia management? A review of the evidence, Curr Opin Lipidol, 2020, 31: 94-100).

Still, the impressive results of the recent REDUCE-IT trial (described by Bhatt D L, Steg P G, Miller M, Brinton E A, Jacobson T A, Ketchum S B, Doyle R T Jr, Juliano R A, Jiao L, Granowitz C, Tardif J C, Ballantyne C M; REDUCE-IT Investigators. Cardiovascular risk reduction with icosapent ethyl for hypertriglyceridamia N Engl J Med. 2019 Jan. 3; 380(1):11-22. doi: 10.1056/NEJMoa1812792), using a high intake (4 g/day) of EPA icosapent-ethyl ester in high-risk patients with high TG concentration and receiving optimal statin treatment, strongly suggest a need to increase the administered dosage, but also to carefully consider the selection of specific n-3 VLCFA molecules. Recent publications suggest an advantage for EPA and its derivatives for improving protection against heart ischemic accidents (previously quoted Mason R P, Libby P and Bhatt D L: Emerging mechanisms of cardiovascular protection for the omega-3 fatty acid eicosapentaenoic acid, Arteriosclerosis, Thrombosis and Vascular Biology, 2020, 40: 1135-1147, doi 10.1161). On the other hand, DHA (and its derivatives) seems to better protect the brain, and possibly the spinal cord and nerves, after traumatic or ischemic injury via a preservation of mitochondrial function (Deckelbaum R J & Calder P C: Is it time to separate EPA from DHA when using omega-3 fatty acids to protect heart and brain? Curr Opin Clin Nutr & Metabol Care 2020, 23: 65-67; (Mayurasakorn K, Niatsetskaya Z V, Sosunov S A, et al (2016): DHA, but not EPA emulsions preserve neurological and mitochondrial function after brain hypoxia-ischemia in neonatal mice. PLoS ONE 11: e0160870. doi 10.1371).

The route of administration may be another important factor in the efficacy of n-3 VLCFA incorporation in cells and their protection against undesirable effects. Indeed, experimental studies in animals and clinical studies in man have shown oral or enteral delivery of FO to be associated to a slow hydrolysis of n-3 FA-containing TG by the pancreatic lipase and low rates of n-3 VLCFA intestinal absorption. In addition, n-3 VLCFA incorporation in cell membranes of different organs and tissues is also slow. This may imply that several weeks of supplementation are needed before observing substantial enrichment in cell membranes. Strategies are developed to improve the bioavailability of orally administered n-3 VLCFA, see for example Maki K C, Dicklin M R: Strategies to improve bioavailability of omega-3 fatty acids from ethyl ester concentrates, Curr Opin Clin Nutr Metab Care, 2019, 22: 116-123.

Of interest, by-passing the gastro-intestinal tract and infusing FO-containing emulsions intravenously may somehow facilitate n-3 VLCFA delivery to cell membranes (Delodder F, Tappy L, Liaudet L, Schneiter, P, Perrudet C, Berger M M: Incorporation and washout of n-3 PUFA after high dose intravenous and oral supplementation in healthy volunteers, Clin Nutr ((2015) 34: 400-8. doi: 10.2016). Still, the described procedure required several infusions of FO emulsions. Such infusions, administered peri-operatively to patients undergoing elective cardiac surgery, did attenuate the post-operative inflammatory response, as shown by a more limited rise of interleukin-6 and interleukin-8. (Berger M M, Delodder F, Tozzi P, Chiolero R L, Tappy L: Three short perioperative infusions of n-3 PUFAs induced by cardiopulmonary bypass surgery: a randomized controlled trial, Am J Clin Nutr, 2013, 97(2): 246-254). This has been confirmed in several reports and meta-analyses evaluating the effect of FO-containing emulsions in surgical patients. Among other benefits, protection against post-operative insulin resistance and reduced loss of muscle mass enabling shorter hospital stay and improved recovery of physical ability have been underlined (Mc Glory C, Calder P C, Nunes E A: The influence of omega-3 fatty acids on skeletal muscle protein turnover in health, disuse, and disease, Front Nutr, 2019, 6: 144 doi:10.3389/fnut 2019.0014)

Medium-chain fatty acids (MCFA) present in medium-chain triglycerides (MCT) have a chain length ranging between 6 and 12 C atoms. The major ones are C8:0 and C10:0, classically found in the liquid phase of coconut oil. In contrast to the long-chain saturated FA, these MCFA are more water-soluble and only partly bound to albumin in plasma. Following oral intake, MCFA's are rapidly digested and absorbed in the intestinal lumen; they are then largely transported to the liver via the portal circulation. Their uptake by cells of several organs (liver, heart, muscle, . . . ) does not require the presence of FA-transport proteins or plasma membrane translocase, and their transport into the cytosol may function without FA-binding proteins. Some MCFA may also cross the blood-brain barrier, such as described by Spector R, Fatty acid transport through the blood-brain barrier, J Neurochem, 1988, 50: 639-643.

In contrast to FA with longer carbon chains, MCFA such as caprylic acid (C8:0) can permeate into the inner mitochondrial space without binding to carnitine. Rapid β-oxidation in mitochondria and cytosolic peroxysomes explains why they are considered as readily available energy substrates (Schönfeld P & Wojtczak L, Short- and medium-chain fatty acids in energy metabolism: the cellular perspective, J Lipid Res 2016, 57:943-954). In the liver, β-oxidation of caprylic acid is five times faster than that of oleic acid. This is associated to glucose sparing and maintenance of glycogen storage. Especially in conditions of low insulin concentration, rapid β-oxidation in the liver produces large amounts of acetyl-CoA, which is partly converted into aceto-acetate and β-hydroxybutyrate, generally referred to as ketone bodies or ketones. Ketones are important fuels for the brain and also a preferred fuel for the heart; as a consequence, caprylic acid (C8:0) may alleviate poor heart ischemic tolerance in CD 36-deficient mice (Labarthe F, Gelinas R, Des Rosiers C: Medium-chain fatty acids as metabolic therapy in cardiac disease. Cardiovasc Drugs Ther, 2008, 22: 97-106). Ketones are also readily available fuels for the muscles (where they maintain or restore a high sensitivity to insulin) and for immune cells. Ketogenic diets have become popular to help obese subjects induce weight loss via an inhibition of triglycerides synthesis in adipose tissues while preserving muscle mass. Apart from those important metabolic roles, MCFA are recognized as agonists of peroxisome proliferator-activated receptors, thereby inducing anti-inflammatory effects. They also play an important role in intracellular signaling and contribute to the regulation of cell metabolism and the control on cell death and survival.

MCT have been essentially used by oral route in patients with severe intestinal malabsorption caused by different etiologies. In addition, in a recent study in weaning piglets challenged with E. coli lipopolysaccharide (endotoxin) the group fed MCT showed a marked protection of intestinal integrity and barrier function (in Xu X, Chen S, Wang H, et al Medium-chain TAG improve intestinal integrity by suppressing toll-like receptor 4, nucleotide oligomerization domain proteins and necroptosis signaling in weanling piglets challenged with lipopolysaccharide Br J Nutr, 2018, 119: 1019-1028).

Besides their ingestion in the gastrointestinal tract, MCT have also been included since more than thirty years in mixed lipid emulsions used in parenteral nutrition. However, the metabolism of MCT administered intravenously markedly differs from that of oral/enteral MCT, since emulsion particles are delivered to all organs, and not essentially to the liver. In this process, a substantial proportion of MCFA are released in the capillary bed after TG hydrolysis by endothelial-bound lipoprotein lipase.

When present in mixed MCT/long-chain soybean-derived triglycerides (LCT) particles, the proportion of MCT that partitions in the particle phospholipid (PL) surface is about four times higher than that of LCT (11 mol % vs. 3 mol %). (Deckelbaum R J, Hamilton J A, Moser A, Bengtsson-Olivecrona G, Butbul E, Carpentier Y A, Gutman A, Oliveira T, Medium-chain versus long-chain triacylglycerol emulsion hydrolysis by lipoprotein lipase and hepatic lipase; Biochemistry, 1990, 29: 1136-42). This increased solubility of MCT in particle surface facilitates access to endothelial-bound lipoprotein (LpL) and hepatic lipases; this explains the more rapid and higher lipolysis of MCT versus LCT molecules, resulting in a much higher release and delivery of MCFA to various tissues. MCFA released by MCT hydrolysis are an excellent source of fuel calories for most organs (see for example Wanten G J, Naber A H: Cellular and physiological effects of medium-chain triglycerides, Mini Rev Med Chem, 2004, 4: 847-57).

Nuclear magnetic resonance studies have shown a much higher solubility for MCT than for LCT in phospholipid bilayers, used as models of cell membranes. MCT also modulate the phospholipid bilayer organization, increasing phospholipid mobility in membranes and resulting in more fluid and more porous membranes, while keeping their carbonyl groups close to the water interface (Hamilton J A, Vural J M, Carpentier Y A, Deckelbaum R J: Incorporation of medium chain triacylglycerols into phospholipid layers: effect of long chain triacylglycerol, cholesterol and cholesteryl esters, J Lipid Res, 1996, 37: 77-782).

Since many lipid molecules—e.g., the long-chain FA—are not soluble in water, emulsions have been developed, which consist of particles having a mean diameter size of 200-280 nm and made of a TG core emulsified by a surface of PL, generally derived from egg-yolk, preferably enriched with antioxidant lipid-soluble vitamins.

Lipid emulsions are essentially used to provide patients requiring parenteral (intravenous) nutrition with a mix of FA and lipid-soluble vitamins. Emulsions are generally included in parenteral nutrition bags and slowly infused together with amino acids, glucose, and several micronutrients.

Various, sometimes severe, side-effects were reported in the USA with the first lipid emulsion based on cotton-seed oil. The first well-tolerated lipid emulsion has been Intralipid® (Fresenius-Kabi), which was first developed in Sweden in the 1960's. It is made of soybean oil emulsified with purified egg-yolk PL. This emulsion is still widely used in several countries. However, soybean oil has a high content of stigmasterol which may be hepatotoxic by antagonizing the bile acid nuclear receptor FXR (Carter B A, Taylor O A, Prendergast D R, et al: Stigmasterol, a soy lipid-derived phytosterol, is an antagonist of the bile acid nuclear receptor FXR, Pediatr. Res., 2007, 62: 301-6). It is also rich in essential LCFA, namely from the n-6 series, with 51-55% C18:2 n-6 vs. a much lower content (6-9%) of C18:3 n-3). Such high content of polyunsaturated FA requires the presence of extra anti-oxidant, generally α-tocopherol, to prevent lipid peroxidation. In addition, the much higher content of n-6 versus n-3 C18 VLCFA favors the elongation-desaturation of the n-6 versus n-3 series, leading to a higher concentration of arachidonic acid (AA) in cell membranes and a depletion of the most active n-3 VLCFA: EPA, DPA, and DHA. Such changes of VLCFA profile in cell membranes are associated to a risk for developing liver steatosis, as well as severe inflammatory reactions, peroxidative damage, and impaired immune defenses. This may be particularly deleterious in neonates and pediatric patients, and in surgical, trauma, septic, and ICU patients.

This has led to the development of lipid emulsions with a reduced content of soybean oil. This was achieved by mixing soybean oil with different oils, for example coconut oil rich in MCT, or olive oil rich in oleate (C18:1 n-9), and more recently by including FO to directly provide n-3 VLCFA. Essentially all these preparations contain 20 g TG and 1.2 g PL derived from egg-yolk/100 ml.

An emulsion exclusively based on FO (Omegaven®, Fresenius), has been marketed since 1998 in Europe. In contrast to other emulsions, Omegaven® contains (only) 10 g TG (of FO) and 1.2 g PL/100 ml and its n-3 VLCFA content is limited (U.S. Pat. No. 9,575,572 B2, M. Lewis: Intravenous omega-3 fatty acid compositions and method of use, 2017). It is well tolerated and claimed to improve liver function tests in infants and children having undergone severe hepatic alterations caused by long-term parenteral nutrition with a soybean oil emulsion. However, plasma elimination, or clearance, rate of this emulsion containing exclusively FO is fairly low (Treskova E, Carpentier Y A, Ramakrishnan R, Al-Haideri M, Seo T and Deckelbaum R J: Blood clearance and tissue uptake of intravenous lipid emulsions containing long-chain and medium-chain triglycerides and fish oil in a mouse model, JPEN 23 (5): 253-7, 1999), as anticipated by previous reports of slow lipolysis by LpL and hepatic lipase for emulsions containing more than 20% FO (Oliveira F L, Rumsey S, Schlotzer E S, et al, Triglyceride hydrolysis of soy oil vs. fish oil emulsions, J Parent Ent Nutr, 21: 224-9, 1997). A potential adverse effect of this slow clearance is the accumulation of emulsion TG in the plasma leading to excessive concentrations of plasma TG. Hence, low infusion rates should be maintained in order to avoid complications, such as increased tendency for coagulation and thrombus formation, higher inflammatory reactions and depressed immune responses.

The intravascular metabolism of lipid emulsions is characterized by two different, and partly interlinked steps:
1) The lipolysis of triglyceride molecules by the enzyme LpL, which takes place at the endothelial site of capillary vessels, releases two free fatty acid (FFA) and one 2-monoglyceride molecules per hydrolyzed TG molecule. A proportion of released FFA is taken up by the adjacent tissue while another proportion enters the circulation and is largely bound to albumin. These FFA will be removed by different organs and tissues. This lipolytic process reduces particle size to form emulsion remnant particles (or remnants).
2) The remnant particles are directly taken up after binding to cell receptors, mainly in the liver but also in several other organs. Such direct particle uptake represents an important pathway for delivering emulsion triglyceride fatty acids and lipid-soluble vitamins into tissues, and is particularly important for fatty acids resistant to release by LpL-mediated TG hydrolysis (step 1).

The clearance, i.e. the elimination from the blood circulation of exogenous TG provided by emulsion injection is an important parameter since high rates injections may substantially raise plasma TG concentrations. Plasma clearance rate of an emulsion can be assessed by measuring TG decay over one hour following the highest concentration measured at the end of a bolus injection of exogenous TG. In addition, if TG measurements on samples taken at sixty minutes post-injection of the emulsion indicate a return of plasma TG concentration to basal (pre-injection) level, this corresponds to a substantially complete clearance of the emulsion. In contrast, no return to basal TG concentration value would indicate exogenous TG accumulation, due to saturation of clearance pathways. Of particular interest are measurements following repeated injections of exogenous TG, since they are more likely to detect exogenous TG accumulation (or not).

In other subsequent studies, TG clearance could be measured after repeated injections in order to test whether such repetitions would lead (or not) to exogenous TG accumulation indicating a saturation of the clearance process. As previously noted for TG hydrolysis (cf supra Treskova et al), the 100% FO emulsion is slowly cleared from plasma and should not be infused at high rates or (a fortiori) administered via bolus injections.

The inventors observed that MCT present in emulsion particle core are largely and rapidly lipolyzed by LpL, which releases a major proportion of emulsion MCFA to be taken up by adjacent tissues (In vivo handling and metabolism of lipid emulsions, Carpentier Y A, Deckelbaum R J, World Rev Nutr Diet. 2015; 112:57-62. doi: 10.1159/000365431. Epub 2014 Nov. 24). These processes markedly reduce the MCT content in the remnants. In sharp contrast, n-3 VLCFA EPA and DHA present in emulsion TG are quite resistant to LpL hydrolysis, and remain in relatively high concentration in remnant particles. Such remnants, depleted of MCT and relatively enriched in n-3 TG, are efficiently taken up via direct particle uptake processes involving various cell receptor and non-receptor mediated pathways (Qi K, Seo T, Al-Haideri M, Worgall T S, Vogel T, Carpentier Y A, and Deckelbaum R J: Omega-3 triglycerides modify blood clearance and tissue targeting pathways of lipid emulsions. Biochemistry 41:3119-3127, 2002). It was further noted that in cells, n-3 VLCFA-containing TG are promptly hydrolyzed by intracellular lipases and a substantial proportion of released n-3 VLCFA is incorporated into PL forming extra- and intracellular membranes.

The present invention is based on the surprising observations that a high proportion of MCT together with a source of n-3 VLCFA in an isotonic intravenous lipid emulsion, not only accelerates the clearance of the infused TG, but also markedly facilitates the rapid delivery and incorporation of n-3 VLCFA in an organ of a human or animal body suffering from a disease, respectively protects that organ against the consequences of an occurred damage. In addition, further experiments indicate that the application of such emulsion on the skin of a human body also facilitates the incorporation of n-3 VLCFA in the epidermis, paving the way to provide prophylactic protection or to boost repair of the skin after different types of injury.

A purpose of the invention is to enable a rapid delivery of n-3 VLCFA and their efficient incorporation in cell membranes of organs following an intravenous bolus injection of rapidly cleared lipid emulsions and possibly a repetition of such injection(s) in patients suffering from different types of acute conditions. The intravenous bolus injection may be repeated at time intervals during at least that day at which the disease occurred, respectively before a severe damage occurs. The used isotonic lipid emulsions have a high content of MCT as they comprise 78 to 95% by weight of the TG content. The high rate of MCT hydrolysis compensates for the slow lipolysis of TG containing n-3 VLCFA and guarantees a fast plasma clearance of the emulsion. The synergistic effect obtained by repeating over time bolus injections of the isotonic lipid emulsion with its high MCT content substantially accelerates the intravascular metabolism of MCT/FO lipid emulsions and the incorporation of n-3 VLCFA in cell membranes.

Of relevance, no significant adverse effects have been observed in any animal or human study evaluating the benefits and risks of bolus injections using the Prontomega® emulsion.

It should be noted that U.S. Pat. No. 9,675,572 B2 describes intravenous pharmaceutical compositions containing omega-3 fatty acids and methods of treating traumatic brain injury, traumatic spinal cord injury and/or stroke using such pharmaceutical compositions. This patent describes emulsions containing at least one omega-3 essential fatty acid, selected from a group consisting of α-linolenate (or ALA), EPA, and DHA, and at least one MCT, wherein the omega-3 essential fatty acids are in a concentration ranging from about 50% to about 90% of the oil phase. One can deduct that the proportion of MCT is from about 10% to about 50%. There are however substantial differences with the present invention wherein the oil phase contains 78-95% MCT and 5-22% FO or other sources of n-3 VLCFA. The hydrolysis of TG by lipoprotein lipase (LpL) and hepatic lipase is considerably lower for FO vs. soybean oil emulsions and the release of n-3 VLCFA from FO is slower than that of the other FA present in emulsion TG. Indeed, the presence of 20% or more FO in the oil phase of mixed TG emulsions substantially slows lipolysis by LpL and hepatic lipase (Oliveira F L, Rumsey S, Schlotzer E S, Hansen I, Carpentier Y A, Deckelbaum R J, Triglyceride hydrolysis of soy oil vs. fish oil emulsions, J Parent Ent Nutr, 21: 224-9, 1997).

The invention will hereunder be described in more details with reference to the drawings. In the drawings:

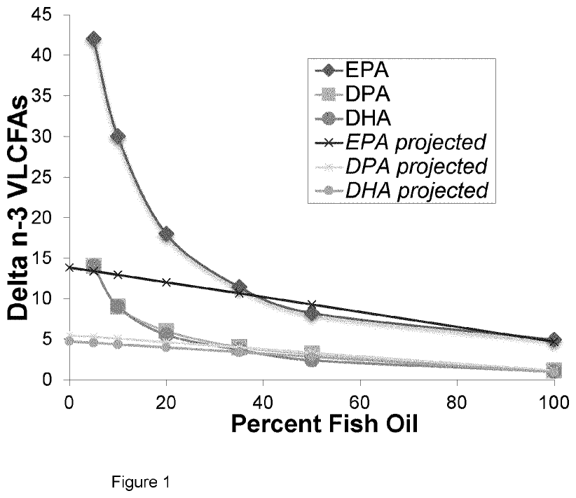

FIG. 1 illustrates the efficacy of omega-3 fatty acid incorporation into the membranes of cultured human umbilical vein endothelial cells (HUVEC);

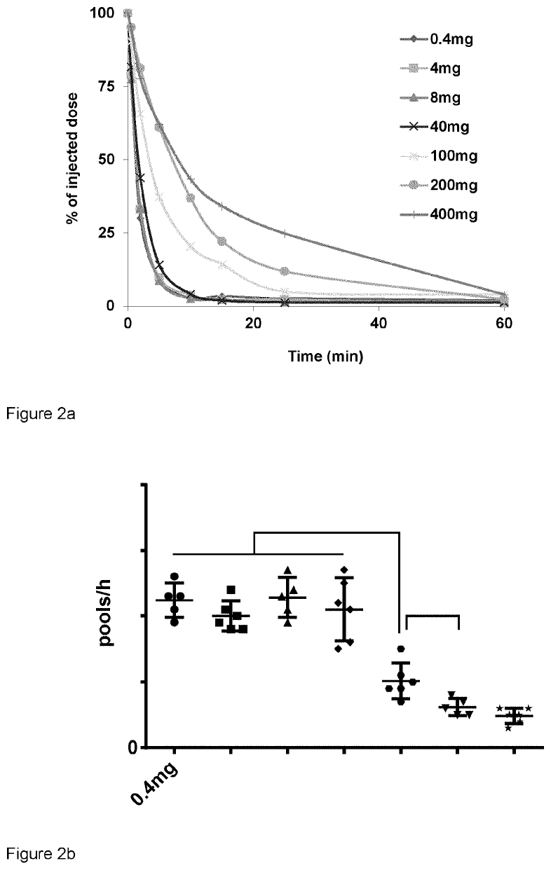

FIGS. 2a and b illustrate the blood clearance decay as a function of the administered doses of Prontomega to rats;

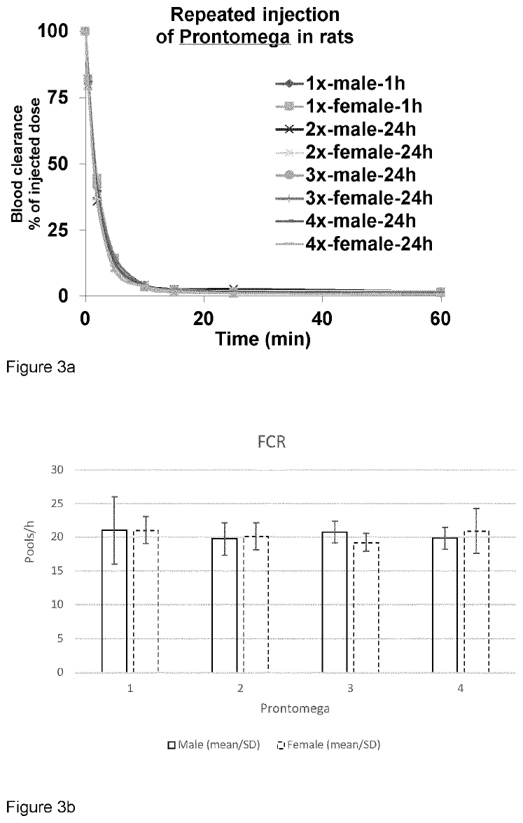

FIGS. 3a and b illustrate the blood clearance after repeating up to four times the doses of Prontomega 40 mg in rats;

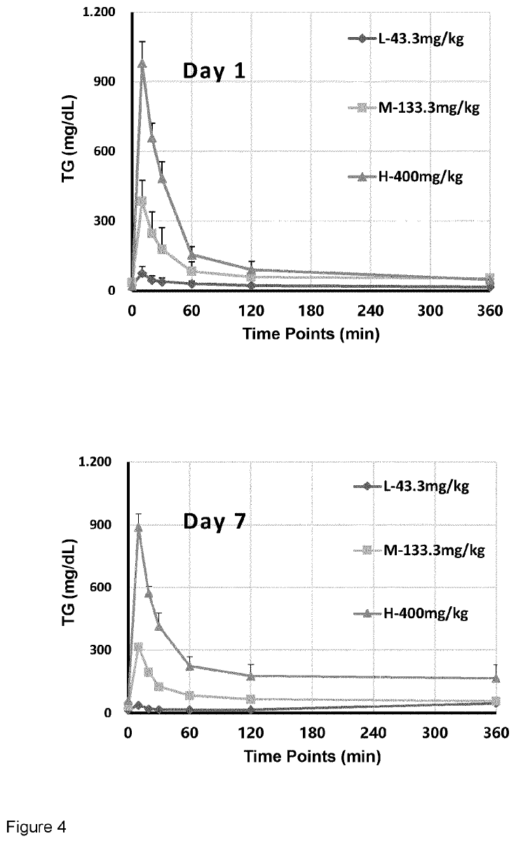

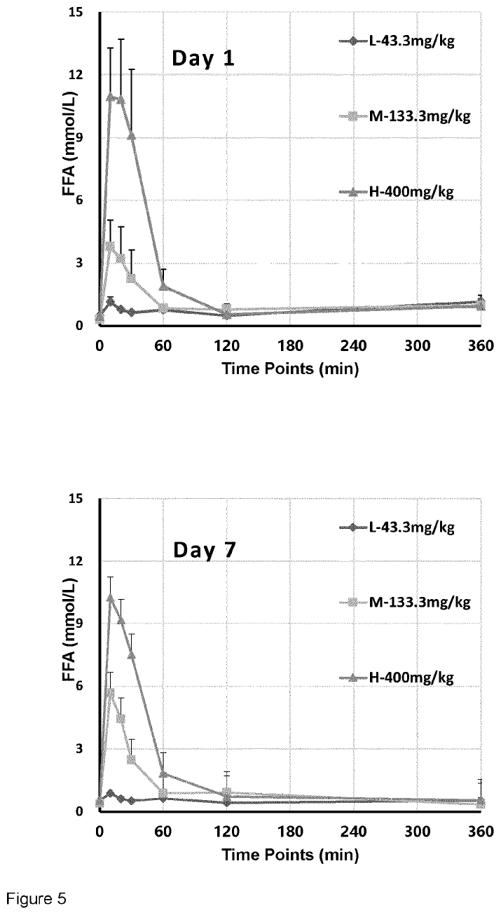

FIGS. 4 and 5 illustrate the decay of plasma TG and FFA concentrations, respectively, in function of the time and following a bolus injection in cynomulgus monkeys;

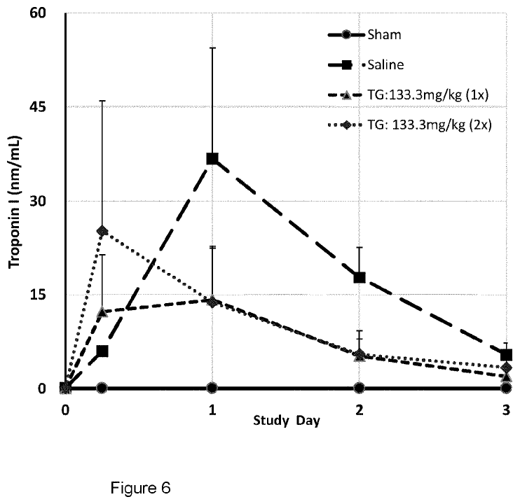

FIG. 6 shows the evolution of the Troponin level over subsequent days post-cardiac ischemia in cynomulgus monkeys;

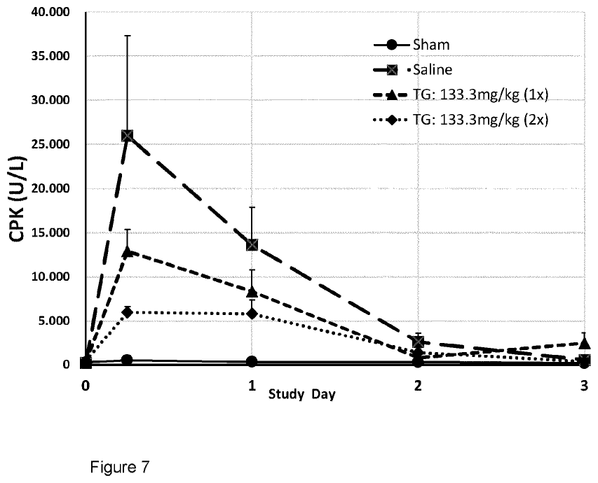

FIG. 7 shows the evolution of the CPK level over subsequent days post-cardiac ischemia in cynomulgus monkeys;

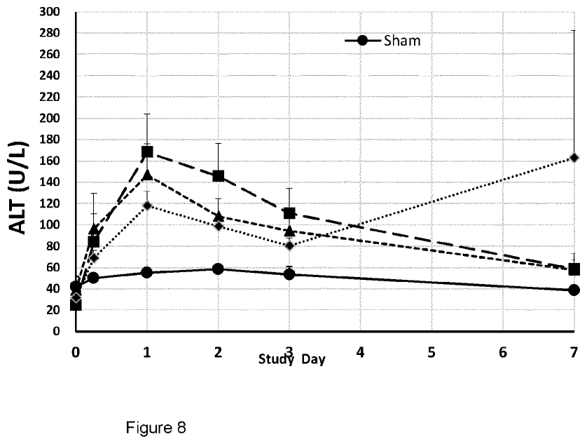

FIG. 8 shows the evolution of the ALT level over subsequent days post-cardiac ischemia in cynomulgus monkeys;

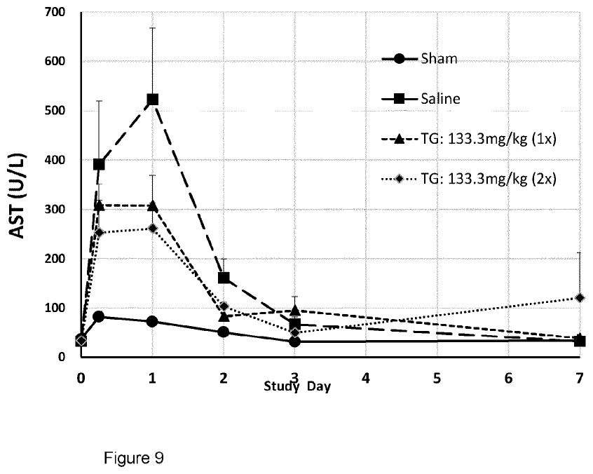

FIG. 9 shows the evolution of the AST level over subsequent days post-cardiac ischemia in cynomulgus monkeys;

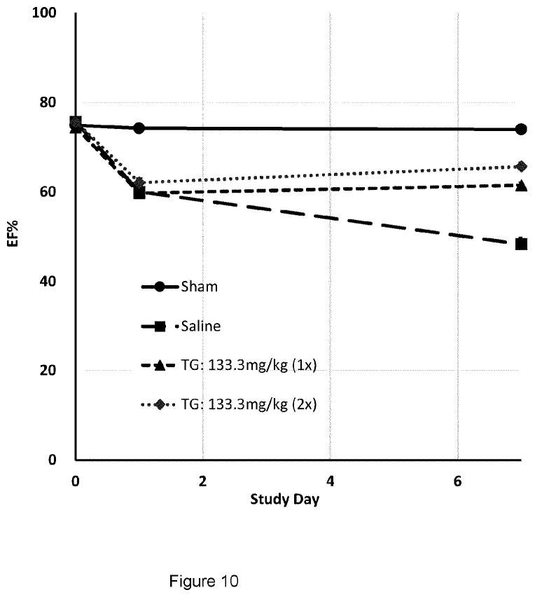

FIG. 10 shows the evolution of the ejection EF on days +1 and +7 post-cardiac ischemia in cynomulgus monkeys;

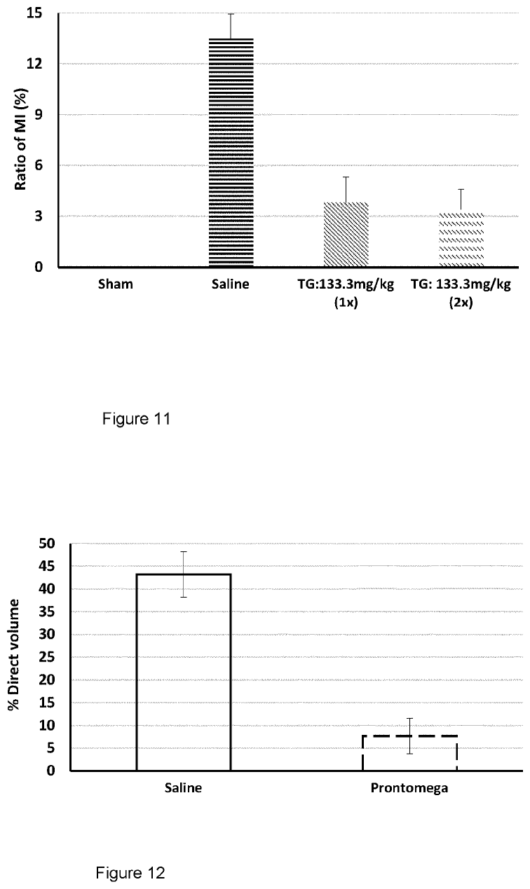

FIG. 11 shows the percentage of infarcted heart area after one week over subsequent days post-cardiac ischemia in cynomulgus monkeys;

FIG. 12 shows the reduction of the infarcted brain volume in a neonatal mouse model of stroke; and

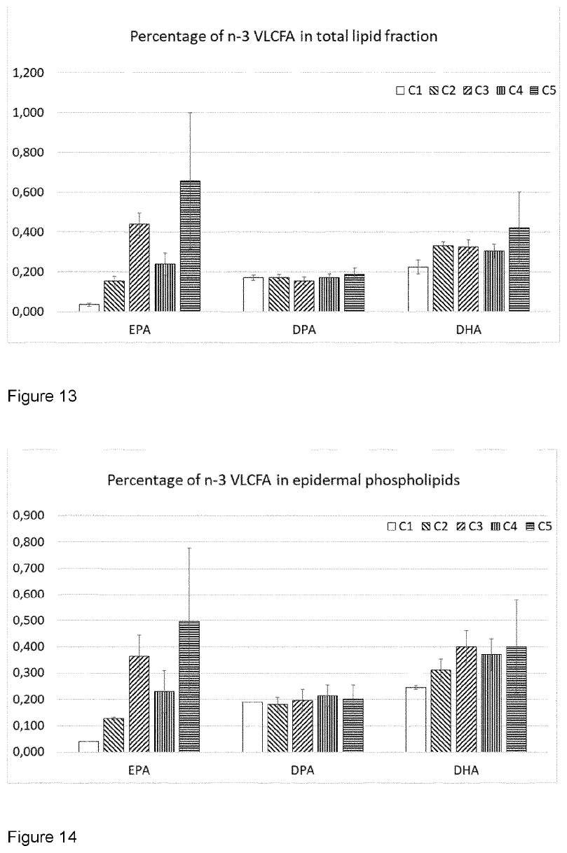

FIGS. 13 and 14 show the enrichment of EPA and DHA in the total lipid and in the phospholipid fractions of the epidermis after topical applications of Prontomega® on the skin.

The aim of the invention is to describe methods of efficient administration of novel lipid emulsions for rapidly providing n-3 VLCFA to key organs, in order to protect patients from the deleterious consequences of acute conditions caused by various etiologies and affecting different organs. Such aim requires a facilitated n-3 VLCFA incorporation in cell membranes as well as, for intravenous administration, an efficient emulsion blood clearance allowing for rapid infusion rate or even bolus injection(s). To this aim, a number of in vitro and in vivo studies in different animal models and in healthy human volunteers were conducted.

Based on observations that the presence of MCT in mixed lipid emulsions may counteract the slow elimination of FO, several preparations with different FO to MCT ratios were tested in vitro: 100% FO/0% MCT; 50% FO/50% MCT; 35% FO/65% MCT; 20% FO/80% MCT; 10% FO/90% MCT, and 5% FO/95% MCT. Since endothelial cells are the first ones to be in contact with the supplied emulsion particles after intravenous administration, these emulsions were supplemented (50 mg/mL) to the medium of cultured human umbilical vein endothelial cells (HUVEC). Concentration of n-3 VLCFA was measured in cell membranes phospholipids, before and after a 4 h incubation with each emulsion.

Surprisingly, decreasing the proportion of FO and increasing the proportions of MCT had little influence on absolute EPA, DPA, and DHA enrichment in HUVEC membrane PL, even when the FO content in the emulsion TG was reduced to 20% by weight. Furthermore, when the EPA and DHA enrichment in HUVEC cell PL was expressed in relation to the FO concentration in the medium, the efficacy for incorporating these n-3 VLCFA was markedly increased by the presence of 80-95% MCT (together with 5-20% FO) in the emulsions.

FIG. 1 illustrates the efficacy of n-3 VLCFA incorporation into the HUVEC membranes, expressed by the increase (delta) in omega-3 fatty acid concentration present in phospholipids divided by the amount of supplied fish oil TG (in function of the percentage of fish oil in the emulsion). As can be seen in this FIG. 1, EPA, DPA and DHA enrichment in cell phospholipid fractions was not proportional to the amount of supplied FO. Accordingly, a substantial relative enrichment was observed even with 5% fish oil in total TG content of the emulsions.

Efficacy of incorporation was much higher for emulsions with FO concentrations less than or equal to 20% by weight and MCT concentrations more than or equal to 80% by weight as illustrated in FIG. 1. The results of this study, made in an ex vivo model, suggest an important and rapid effect of MCT in contact with cells to increase the capacity of cell membranes to accommodate higher concentrations of n-3 VLCFA.

The 20% by weight of fish oil and 80% by weight medium chain triglycerides emulsion mixture was selected for further in vivo studies. This preparation, which also contains egg-yolk derived PL (1.2 g/dl) and glycerol (2.5 g/dl), as well as an adequate supplement of alpha-tocopherol (60 mg/dL), has received the brand name Prontomega® and will hereinafter be referred to as such.

Several experimental studies using Prontomega® were then conducted in various animal models, and namely in animals depleted of n-3 VLCFA to mimic conditions prevailing in many human populations. These experiments have confirmed that depletion of n-3 VLCFA is associated to peripheral insulin resistance in muscles, adipocytes, and liver, as well as to a substantial degree of steatosis (Carpentier Y A, Portois L. and Malaisse W J, N-3 fatty acids and the metabolic syndrome, Am J Clin Nutr 83(suppl) 1499S-1504S, 2006).

In another study using the ex vivo Langendorff model of cardiac ischemia in n-3 FA depleted rats, decreased basal cardiac function as well as impaired recovery were observed following ischemia-reperfusion (Perturbation of phospholipid and triacylglycerol fatty acid positional location in the heart of rats depleted of n-3 long-chain polyunsaturates. Portois L, Peltier S, Sener A, Malaisse W J, Carpentier Y A. Nutr Res.

In these n-3 FA depleted rats, a single bolus injection of Prontomega® promptly induced a rapid and substantial enrichment of DHA in liver and brain cell PL, and of EPA in PL of other organs, i.e. within the first samples taken at one or two hours post-injection (Peltier S, Portois L, Malaisse W J and Carpentier Y A, Preferential enrichment of liver phospholipids in docosahexaenoate relative to eicosapentaenoate in omega-3-depleted rats injected with a medium-chain triglyceride: fish oil emulsion, Prostaglandins Leukot Essent Fatty Acids, 78(1):27-32, 2008.). Such bolus injections of Prontomega corrected many metabolic alterations associated to n-3 VLCFA depletion in rats and mice (Carpentier Y A, Peltier S, Portois L, Sebedio J L, Leverve X, Malaisse W J Rapid reduction of liver steatosis in omega3-depleted rats injected with a novel lipid emulsion, Horm Metab Res.; 40(12):875-879, 2008). Globally, these studies made using in vivo animal models, showed improvement in the different features of the metabolic syndrome but also a protection against the consequences of cardiac ischemia in n-3 VLCFA depleted rats (Peltier S, Malaisse W J, Portois L, Demaison L, Novel-Chate V, Chardigny J M, Sebedio J L, Carpentier Y A, Leverve X M, Acute in vivo administration of a fish oil-containing emulsion improves post-ischemic cardiac function in n-3-depleted rats Int J Mol Med. 18(4):741-749, 2006). These animal studies using bolus injections of Prontomega also suggested high levels of safety and tolerance for this mixed 20% FO/80% by weight MCT emulsion, when injected in bolus.

A study was then conducted in twelve human volunteers who were randomized to receive an injection of 50 mL of either a control emulsion with 50% LCT (soybean oil)/50% by weight MCT, or the 20% FO/80% MCT Prontomega® emulsion. After an eight week wash out period, the subjects received another injection of the other preparation than the one they had received earlier. The rate of plasma clearance was higher for the 20% FO/80% MCT Prontomega® preparation than for the 50% LCT/50% by weight MCT emulsion, and a substantial enrichment of EPA was already observed in WBC- and platelet PL in the first blood sample drawn at 60 min post-injection for analyses of individual FA concentrations. No clinical side effects were noticed and all biological parameters of organ tolerance and function and of blood coagulation remained within normal value ranges. Detailed descriptions of this study are published (Carpentier Y A, Hacquebard M, Portois L, Dupont I E, Deckelbaum R J, Malaisse W: Rapid cellular enrichment of eicosapentaenoate after a single intravenous injection of a novel medium-chain triacylglycerol:fish-oil emulsion in humans, Am J Clin Nutr. 2010 April; 91(4):875-82); Pradier O, Portois L, Malaisse W J, Carpentier Y A: Hemostatic safety of the bolus intravenous injection of a novel medium-chain triglyceride:fish oil emulsion, Int J Mol Med. 2008 September; 22(3):301-7).

In conclusion, the Prontomega® emulsion combines optimal dosage of several very active ingredients:
a refined fish oil emulsion providing n-3 VLCFA EPA, DPA, and DHA; when present in fish oils, these n-3 VLCFA molecules modulate the inflammatory reactions, reduce insulin resistance and peroxidative damage as well as risk of thrombosis, sustain endothelial function and tissue micro-perfusion, and rapidly favor healing processes. However, each of these VLCFA molecules may be more appropriate than the two others to treat specific pathological conditions in specific organs, hence, the inventors also propose instead of the FO component of the emulsion, the same proportion (5-22% per weight) of distinct n-3 VLCFA molecules, alone or in different combinations;
a substantial proportion of MCT: their action in PL surface of emulsion particles is to accelerate emulsion clearance and therefore allow for repeating bolus injections several times; moreover, their presence in cell membrane PL may modify the physical properties of cell membranes and markedly facilitates n-3 VLCFA incorporation in key organs; additionally MCT provide efficient energy uptake to all organ cells, either directly as MCFA or indirectly as ketone bodies produced in the liver. MCFA are also active in modulating inflammatory reactions and immune responses (The addition of medium-chain triglycerides to a purified fish oil-based diet alters inflammatory profiles in mice. Carlson S J, Nandivada P, Chang M I, Mitchell P D, O'Loughlin A, Cowan E, Gura K M, Nose V, Bistrian B R, Puder M. Metabolism. 2015 February; 64(2):274-82. doi: 10.1016/j.metabol.2014.10.005. Epub 2014 Oct. 13.);
a content of alpha-tocopherol, well in excess of that required to protect emulsion VLCFA from peroxidation; some of this extra content will be transferred to circulating lipoproteins; some will be delivered and stored in the liver, and some may also modulate inflammatory reactions and their consequences.

Several studies of pharmaco-dynamics and pharmaco-kinetics have been conducted by the inventors in murine models and in monkeys, using different dosages as well as repeated bolus injections of Prontomega®.

Prontomega®, labelled with ³H-cholesteryl ester (CE) was injected at doses of 0.4, 4, 8, 40, 100, 200, and 400 mg in fed and fasted, male and female rats (n=6 animals for each group). ³H-CE was measured in blood at 0, 0.5, 2, 5, 10, 15, 25 and 60 min post-injection. Animals were sacrificed after the 60 min blood drawing and ³H-CE measured in several organs such as the brain, heart, lung, liver, kidney, spleen, visceral and subcutaneous fat, muscle and bone. As shown in FIG. 2a the blood clearance decay of ³H-CE was exponential for all doses. As shown in FIG. 2b the FCR was highest and not different for doses 0.4 up to 40 mg. The blood clearance rate and FCR gradually decreased for doses ranging between 100 and 400 mg. The FIGS. 2a and b thus teach that increasing the doses to 100 mg and more for a single injection adversely affects the blood clearance. ³H-CE blood clearance rates were not different between fed and fasted, and between male and female rats.

With respect to tissue distribution of Prontomega® at 60 min post-injection the organ uptake was predominant in the liver, followed by spleen and muscle (with no dose effect), and heart and lung (again, with no dose effect). There was no difference observed between male and female animals.

In a subsequent study, injections of Prontomega® at the medium dose of 40 mg were performed either once, twice, three or four times in male and female rats. The Prontomega® injections were repeated at hourly intervals. Blood clearance kinetics were measured using Prontomega® labelled with ³H-CE as described here before by following ³H-CE decay in blood at 0, 0.5, 2, 5, 10, 15, 25 and 60 min post-injection. In addition, plasma TG and FFA were measured at time 0 h, 1 h, 4 h and 24 h post-injection. Animals were sacrificed after the 24 h blood drawing and ³H-CE measured in several organs, such as brain, heart, lung, liver, kidney, spleen, visceral and subcutaneous fat, muscle and bone.

As shown in FIGS. 3a and 3b the blood clearance decay of ³H-CE and the FCR were not affected by the repetition of 40 mg injections and did not differ between male and female animals. In addition, plasma TG concentrations measured at 1 h and 4 h following the last injection did not show any increase over basal values after repeated injections, indicating complete clearance of the injected TG. This shows that repeating the injections over time, while maintaining the injected doses, in particular at 40 mg, does not adversely affect the blood clearance rate.

With respect to tissue distribution of Prontomega® at 60 min post-injection, organ uptake was predominant in the liver, followed by muscle, spleen, and lung. There was no effect of repeating injections on the TG concentration in the liver, spleen, heart and lung of any group; as well, no increase of ³H-CE was observed in any organ and there was no TG accumulation at 24 h. in the liver in any group, No clinical or biological sign of toxicity was observed after Prontomega® injections in the rats.

Prontomega® was also injected in cynomolgus monkeys. The doses were selected based on previous studies in rodents and humans, with a medium dose of 133.3 mg/kg body weight (b.w.), a low dose of 43 mg/kg b.w. (3 times lower) and a high dose of 400 mg/kg b.w. (3 times higher). The emulsion was daily injected over a 7-day period. Those results are shown in FIG. 4.

Plasma TG and FFA levels were measured at 0, 10, 20, 30 min and at 1 h, 2 h, 6 h, and 24 h post-injection on days 0 and 7. In addition, plasma TG and FFA levels, and platelet counts, were daily monitored. Other biological parameters, such as liver and renal function tests, CRP, blood counts and coagulation parameters, were examined at baseline, i.e. before emulsion injection and at 24 h post the previous injection, and on days 2, 4 and 8. Plasma samples, and separated WBC and platelets, were stored at −80° C. for later FA analyses by GC-mass spectrometry.

As shown in FIG. 4 and FIG. 5, exponential decreases of TG and FFA levels, respectively, were observed from 10 to 60 min, with a return to basal levels reached at 60 min for the low and medium doses. The elimination kinetics were not different, for the three doses. However, while TG concentrations returned to basal values at 2 h post-injection of the low and mid doses, they plateaued at a higher concentration (150 vs. 70 mg) when measured again on day 7, but only for the highest injected dose. Total WBC and platelet (PLT) counts, as well as WBC subset distribution (monocytes and neutrophils), were not affected by daily injection of Prontomega® at any dose over seven days. It was also observed that Prontomega® injections rapidly increased concentrations of MCFA, already 10 min after the injection, as well as of n-3 VLCFA (EPA, DPA, DHA) in the total plasma, in the FFA pools, as well as in platelets. In addition, Prontomega® injections over seven days caused a dose-related increase of EPA, DPA, and DHA content in the liver.

These detailed pharmaco-kinetic studies in two different animal models (rats and monkeys) confirm the very efficient plasma elimination of Prontomega® after bolus injections of low, medium, and high doses of Prontomega®. The results provide evidence for a clearance rate for Prontomega® which is unmet by any other n-3 VLCFA-rich preparation.

Some studies also included kinetic measurements of the elimination and potential TG accumulation in plasma and in organs after repeated bolus injections (in particular up to four injections at 1 h interval in rats and seven consecutive daily injections in monkeys). Kinetic measurements showed no influence for repeated injections on clearance rate, and no TG accumulation in organs for any dosage. TG accumulation in plasma was observed only after repeated injections at the highest dosage. Substantial incorporation of n-3 VLCFA was observed in major organ PL, indicating incorporation in cell membranes.

The results given above indicate that bolus injections of Prontomega® may be repeated after a short time interval, for example within 1 h, which offers a solution to very rapidly deliver n-3 VLCFA to key cells and organs, such as for example to the heart, brain, lung, liver, but also WBC and platelets. This may provide substantial benefits in patients in acute conditions after suffering from organ damage, in particular heart or brain ischemia, as well as in other acute conditions, e.g. before and after severe surgical operations and after trauma, including damage in the central nervous system (CNS) and the spinal cord, and before and after organ transplantation.

A pilot efficacy study was conducted in cynomolgus monkeys to determine whether one or two bolus injections of Prontomega®, performed at 2 h after the induction of a myocardial infarct (MI) and repeated on the 2 following days, could modify parameters of cardiac (dys-)function over the following week and the proportion of infarcted heart area when measured one week later. The cynomolgus monkey (Macaca fascicularis) was selected because it represents a relevant primate model fairly close to human subjects.

Eighteen male cynomolgus monkeys (bodyweight: 3.0-5.0 kg) were selected for the study and underwent an acclimatization period of four weeks. They were individually housed and fed a high-fat diet during the last 2 weeks of acclimatization to better mimic dietary intake in humans.

The animals were randomly allocated to the following groups:
Group A (n=2): sham operation, i.e. a thoracotomy followed by a dissection of the mid-left anterior descending coronary artery but no ischemia (I). The thorax closure (C) was done after 1 h.
Group B (n=4): MI control «saline» group, i.e. thoracotomy, dissection of the mid-left anterior descending coronary artery and ischemia during 1 h prior to reperfusion (R) and thorax closure (C); injection of saline at 1 and 2 h post-reperfusion and again on post-ischemia days 1 and 2.
Group C (n=4): MI Prontomega® “1× injection” group, i.e. thoracotomy, dissection of the mid-left anterior descending coronary artery and ischemia during 1 h prior to R and C, followed by an injection of Prontomega® at 1 h post-reperfusion and 1 injection again on post-ischemia days 1 and 2.
Group D (n=4): MI Prontomega® “2× injection” group i.e. thoracotomy, dissection of the mid-left anterior descending coronary artery and ischemia during 1 h prior to R and C, followed by an injection of Prontomega at 1 h and again at 2 h post-reperfusion, and 2 injections (at 1 h interval) on post-ischemia days 1 and 2.

On day 0, the fasted animals were anesthetized by Zoletil® (intramuscular (IM, 5 mg/kg) and Xylazine (IM, 5 mg/kg), while buprenorphine (IM, 0.2 mg/kg) was used to provide perioperative pain relief. The core temperature, heart rate, respiration rate, ECG and SPO₂ were recorded throughout the surgical procedure. Lidocaine was used prior to ischemia/reperfusion to prevent ventricular fibrillation.

The following biological parameters were considered:

Troponin is released during suffering of myocardial cells and its concentration is classically measured to detect a myocardial infarct and the magnitude of myocardial cell suffering. As expected and illustrated in FIG. 6, troponin concentration did not rise at all in group A, in contrast to the other groups having undergone I/R. Troponin rise tended to be more marked at 6 h post-UR in groups C (12.3±9.2) and D (25.3±20.7) than in saline control group B (6.0±0.4). However, troponin concentrations on post-ischemia day 1 were substantially higher in group B (36.8±17.7) than in groups C (14.2±8.3) and D (13.8±9.1). While a decrease of troponin concentrations was observed on days 2 and 3 in groups B, C, and D, values were quite lower in groups C (5.2±2.7 and 2.0±1.2) and D (5.5±3.8 and 3.4±2.6) than in group B (17.8±4.9 and 5.4±1.9). On days 2, 3 and 4, comparable troponin values were measured in groups C and D injected with Prontomega®.

Cardiac creatine Phosphokinase (CPK) is released after necrosis of myocardial cells. As illustrated in FIG. 7, CPK concentration did moderately rise at 6 h post-UR in group A and progressively returned to basal values, reached on day 3. In contrast, very substantial rises of CPK concentrations were observed at 6 h post I/R in the 3 groups (B, C and D) submitted to MI. However, substantial differences were observed between the 3 groups. Indeed, mean CPK values in group B peaked at 6 h post I/R at 25.98+/−11.34 U/L in group B vs. 12.91+/−2.47 U/L in group C and 5.99+/−68 U/L in group D. CPK concentrations substantially decreased over the following 2 days and were close to basal values in the different groups on day 3.

Enzymes Alanine aminotransferase (ALT) and Aspartate aminotransferase (AST) are released by suffering of hepatic cells; however, an increase of ALT specifically indicates liver cell injury while a rise of AST may also result from muscle and heart injury.

As illustrated in FIG. 8, ALT concentration rose very slightly above normal values in sham group A and were back to basal value on day 8. Substantial rises of ALT concentration were observed in the 3 MI groups: in group B, ALT concentration was elevated at 6 h post I/R, and remained elevated on days 1, 2 and 3; the rise of ALT tended to be less marked in group C, and (namely) in group D.

As illustrated in FIG. 9, AST concentration rose slightly above normal values in sham group A and were back to basal values on day 3. ALT concentration rose substantially more in the 3 MI groups, with a peak on day 1; the peak was lower in groups C and D than in control group B.

Urea and creatinine concentrations, measured on days 0 and 7, did not rise in any group, suggesting the maintenance of normal renal function.

Hemoglobin concentration and hematocrit did decrease by 10-15% from day 0 to day 7 in the sham group A, as well as in the 3 groups undergoing I/R, with no between groups difference.

Platelet count, measured on days 0 and 7, remained unchanged in sham group A, but was increased on day 7 in all animals of I/R groups B, C, and D.

White cell count, measured on days 0 and 7, remained unchanged in sham group A. It was increased on day 7 in 2/4 animals of I/R group B, but was decreased on day 7 in all animals of groups C and D. The proportion of neutrophils, lymphocytes, and monocytes, remained essentially unchanged when measured on day 7 compared to day 0.

C-reactive Protein (CRP) is an indicator of inflammation. CRP concentration did increase in all groups after surgery. However, the increase was very limited in group A. In contrast, a substantial rise of CRP concentration was observed in all groups of animals having undergone I/R, with a peak to values >40 mg/L on day 1. CRP concentrations progressively declined from day 1 to day 7 but remained slightly above basal values.

Fibrinogen is involved in the coagulation pathway, and its concentration increases during inflammatory reactions. Fibrinogen concentration remained essentially unchanged in sham group A. In contrast, a marked elevation of fibrinogen concentration was observed on days 1, 2, and 3 in the 3 groups having undergone MI by I/R. Fibrinogen rise was as marked in Prontomega groups C and D than in group B.

Prothrombin time (PT), activated partial thromboplastin time (APTT), and thrombin time (TT) remained unchanged all over the study period in all groups.

Cardiac ultrasound echography was measured prior to surgery and again on post-operative days 1 and 7. This technique allows to measure left ventricular ejection fraction (LVEF), left ventricular end-diastolic volume (LVEDV), left ventricular end-systolic volume (LVESV), ejection fraction (EF) % and fraction shortening % (FS).

The different parameters measured during echography remained unaffected in group A animals having undergone a sham thoracotomy without I/R. In contrast, substantial changes were observed on day 1 in the groups having undergone I/R.

An increase of the left ventricular internal diameter during diastole (LVIDd) and during systole (LVDs) was observed on day 1 in the three groups having undergone I/R, with no difference between saline group B and Prontomega® groups C and D.

An increase of the left ventricle volume at the end of diastole (LVEDV) and at the end of systole (LVESV) was also observed on days 1 and 7. However, if no difference between saline group B and the Prontomega groups C and D was noted for volumes measured at the end of diastole, the increase in volume at the end of systole was substantially less marked on day 7 in Prontomega groups C and D than in saline control group B.

As illustrated in FIG. 10, ejection fraction (EF) was decreased on day 1 in groups B (−21%), C (−20%), and D (−18%). However, if a further decrease of EF was observed on day 7 in control group B (−36%), EF values remained stable (at −17%) in group C and tended to improve (from −18% to −13%) in group D.

Similarly, a decrease of fraction shortening (FS) was observed on day 1 in the 3 groups B (−30%), C (−28%), and D (−28%). However, if a further decrease (from −30% to −46%) was observed in control group B on day 7, a stabilization was observed in group C (−28% and −25%) and an improvement (from −26% to −20%) was observed in group D.

Hemodynamic measurements performed on day 7 showed lower values for systolic and diastolic pressure in the 3 groups having undergone I/R, with no clear difference between saline group B and Prontomega groups C and D. However, left ventricular pressure at the end of systole was decreased only in saline group B (85+/−8.5 mm Hg), but not in Prontomega groups C (93+/−3 mm Hg) and D (94+/−10 mm Hg) on day 7.

The hearts, as well as other organs, were collected at necropsy performed on post-ischemia day 7, after the echography measurements. The percentage of infarcted heart area (MI size) was measured by staining using TTC/Evans blue.

As can be seen in FIG. 11, the percentage of infarcted heart area was, as expected, 0% in group A. It varied between 11 and 16% (mean: 13.5±1.4%) in group B. In contrast, the infarcted heart area varied between 1.0 and 6.9% (mean 3.8±1.5%) in group C and between 1.0 and 6.0% (mean: 3.4±1.2%) in group D. The reduction of infarcted area between groups C and D having received Prontomega® injections and saline control group B receiving was highly significant (p<0.01).

Heart weight, expressed in relation to body weight, was 0.32+/−0 in sham group A, 0.35+/−0.01% in saline group B, but 0.30+/−0.0% in groups C and D. The difference between group B and groups C and D suggests a protection against cardiac failure provided by Prontomega® injections.

In this pilot study performed in monkeys, ischemia of the mid-left anterior descending coronary artery followed one hour later by reperfusion (I/R model), induced a substantial rise of troponin and of various enzymes that was already present at 6 h post-ischemia. However, levels of different markers of myocardial and liver injury, rapidly became substantially lower in the 2 groups receiving Prontomega®. Similarly, ALT and AST concentration tended to rise less in group C that received single injections of Prontomega®, and significantly less in group D animals where Prontomega® injection was repeated after 1 h interval. Hemodynamic and ultrasound measurements indicate a strong protective effect of Prontomega® injections on cardiac contractile function post-MI, as well as a marked reduction of infarct size. The rise of CRP and fibrinogen levels was not reduced in the two groups injected with Prontomega®. This suggests that the protection provided by Prontomega® injections was not only due to an anti-inflammatory effect, but also, and possibly mainly, via an effect on endothelial and platelet functions. Another major effect of Prontomega® is on preventing cell death after ischemic injury. The clinical and biological parameters indicate no toxicity associated to bolus injections of Prontomega®, even when repeated at hourly intervals.

Another study was performed using the monkey model of cardiac ischemia to determine the most appropriate dosage of Prontomega® to be injected at 2 h post-ischemia. This was done in groups of n=8 animals. Three dosages of Prontomega® were evaluated: 43.3, 133.3, or 400 mg/kg by weight. The control groups consisted of sham operated animals undergoing no ischemia, and 2 groups undergoing cardiac ischemia: a saline control (n=8), and a “positive control” group (n=8) treated with Metoprolol tartrate (3 mg/kg), as usually applied to MI patients.

Enzymes (Troponin and NGAL), indicating cardiac cells suffering, declined faster and to lowest levels on days 2 and 3 in the Prontomega® (133 mg/kg) group. NT Pro BNP concentration (indicating the risk of cardiac failure) remained normal (9.0 pg/mL) in the sham group on day 7, but was increased to 44.0+/−8.3 and 45.7+/−15.4 pg/mL in the saline and in the positive control groups respectively. NT Pro BNP level on day 7 was 32.4+/−7.0, 28.0+/−6.3, and 34.9+/−12.3 pg/mL in the 3 Prontomega® treated (43.3, 133.3, and 400 mg/kg, respectively) groups.

The index of myocardial contractility (−dp/dt) decreased in the saline control and to some extent in the 43.3 mg/kg groups, but remained unchanged in the 133.3 and 400 mg/kg Prontomega® as well as in the Metoprolol tartrate groups.

The ratio of infarcted heart area measured on day 7 was 12.68+/−2.15% in the saline control group vs. 7.73+/−1.61, 4.56+/−1.36, and 4.38+/−1.19% in the 3 Prontomega® groups, and 5.80+/−1.83% in the positive Metoprolol group. The positive Metroprolol group being the group which had received a conventional MI treatment. The results indicate that the (133.3 mg/kg and the 400 mg/kg) Prontomega® groups showed a 64% reduction of infarcted area, and the Metoprolol group a reduction of 55%.

These results confirm that Prontomega® bolus injections post-cardiac ischemia contributes to substantially reduce the ratio of infarcted heart area and induce a faster recovery of cardiac contractility. As noted in previous studies, Prontomega® injections did not induce any significant adverse effect. The results also confirm that the dose of 133.3 mg/kg combines efficient plasma clearance and clinical efficacy, and may be recommended for use in different animal and human bodies.

Another comparison test between saline and Prontomega®, this time administered intra-peritoneally (IP), was performed in 16 C57BL/6J neonatal, 10 day-old, mice from both genders undergoing brain ischemic injury. Ischemia (I) was induced by right carotid artery ligation including cauterization and cutting of the artery; this procedure generally lasted less than 5 min. Ninety minutes after the surgical procedure, the animals were exposed to a 15 min hypoxia (H) under 8% O₂ at 37° C. with reperfusion starting at the end of the hypoxia period when the animals were placed back at room temperature.

The test group (n=9) was injected IP with Prontomega® (375 mg TG/g) immediately after and again at 1 h after hypoxia. The control group (n=7) was injected IP with the same volume of saline at the same time points.

The animals were sacrificed at 24 h after ischemic injury. Brains were immediately harvested and 1-mm thick slices colored with triphenyl-tetrazolium chloride (TTC), 2% in PBS, to evaluate infarct volume. The percentage of ischemic brain hemisphere volume was 43.17+/−5.00 (mean+/−SEM) in the saline control group, vs. 7.68+/−3.89% in the Prontomega test group. As shown in FIG. 12, the mice injected with Prontomega® showed a 82% reduction of ischemic brain volume by comparison to the saline injected control animals.

This study performed in a mouse model (H/I) of neonatal stroke also provides evidence for the substantial efficacy against brain ischemia of the Prontomega® emulsion administered intraperitoneally.

From the tests done on animals it can be derived that for application to a human body suffering from a cardiac ischemia or for whom a serious risk exists that a cardiac ischemia is occurring, bolus injections of preferably 50 ml Prontomega® emulsion should be administered. Such a 50 ml Prontomega® emulsion comprises 10 g of TG, 2 g of FO containing 1.2 g of EPA and DHA. A first administration should be applied either as soon as possible after the cardiac ischemia occurred, or as soon as possible after having established that such a risk exists. A second administration should preferably be done between one and three hours after the first administration, depending on the plasma TG concentration. On the next two days following the first administration, two administrations per day should be done within a time interval of between one and three hours separating the administrations. A similar number of administrations within a similar sequence could be used in case of brain ischemia.

Having realised that the incorporation of n-3 VLCFA in organs like the brain or the heart could be improved in the way described herebefore, the inventors were triggered to investigate if such an incorporation of n-3 VLCFA could also be realised in another important organ, i.e. the human skin, in particular the epidermis layer. This was done using applications of Prontomega® on ex vivo human skin samples. In addition, the impact of repeated applications and of the n-3 VLCFA penetration period on their incorporation was also studied.

The human skin samples were obtained from abdominal surgery of an unique donor. Skin samples of 2 cm² were placed on static cells and maintained at 32°±1° C. for the whole study period. The receptor fluid was PBS 0.01M pH7.4+5% BSA. Twenty mg Prontomega® was applied on the skin surface of each static cell and carefully spread over the diffusion area of the skin sample by performing a massage with a glass rod during 30 seconds. The exact amount of formulation remaining onto the skin, after pipetting and massage, was calculated.

Two static cells (M & N) contained skin samples used as a reference, with no Prontomega® application. They were harvested after 24 h. The results obtained by those cells are indicated by the columns C1 in the FIGS. 13 and 14.

Three applications of Prontomega® were done at hourly interval in in skin samples of cells (A, B, C) which were harvested 1 h after the last application. The results obtained by those cells are indicated by the columns C2 in the FIGS. 13 and 14. Three other applications of Prontomega® were done at hourly interval in skin samples of cells (D, E F), but the penetration period was extended until harvesting at 24 h. The results obtained by those cells are indicated by the columns C3 in the FIGS. 13 and 14.

Six applications of Prontomega® were done at hourly interval in skin samples of cells (G, H, I) which were harvested 1 h after the last application. The results obtained by those cells are indicated by the columns C4 in the FIGS. 13 and 14. Six applications of Prontomega® were also done at hourly interval in skin samples of cells (J, K, L), but the penetration period was extended until harvesting at 24 h. The results obtained by those cells are indicated by the columns C5 in the FIGS. 13 and 14.

For each skin sample, five strips of stratum corneum were collected. Epidermis was separated from dermis using a scalpel blade. Receptor fluid was collected as well as washing fluid. All samples were frozen at −80° C. until fatty acid analyses which were performed within 3 weeks, first in total lipids and afterwards in the phospholipid fraction of lipid layers using gas chromatography coupled to flame ionisation detector (GC-FID) method.

N-3 VLCFA were detected in the epidermis only and not in the dermis and the stratum corneum. No enrichment of DPA was observed in any layer as is shown in the FIGS. 13 and 14.

As shown in FIG. 13, by comparison to reference values (column C1) the 3 consecutive applications of Prontomega® (cells A,B,C; column C2) induced a 4- to 5-fold increase in the EPA concentration and a 50% increase in the DHA concentration in total lipids of the epidermis. Extending the penetration period to 24 h after 3 applications (cells D,E,F; column C3) led to further increase of EPA concentration in total lipids, but no further change of DHA.

The 6 consecutive applications, shown in column C4 of FIG. 13, led to a further rise of EPA concentration by comparison to 3 consecutive applications, but no further enrichment of DHA. Extending the penetration period to 24 h after 6 applications (cells J,K,L; column C5) led to further increases of EPA concentration, and a further enrichment of DHA (cells J,K,L).

FIG. 14 shows changes of n-3 VLCFA in the phospholipid fraction of the epidermis, present in high concentration in cell membranes. Three consecutive applications of Prontomega® (cells A,B,C; column C2) induced a 4-fold increase in the EPA concentration and a 20% increase in the DHA concentration, The 6 consecutive applications (column C4) led to a further rise of EPA concentration (6-fold from control values), and a 50% enrichment of DHA concentration. Extending the penetration period to 24 h after 3 (cells G,H,I; columns C4) and 6 applications (cells J,K,L; columns C5) led to further substantial increases of EPA concentration (9- and >10-fold from control values), but no further enrichment of DHA.

The results of the Prontomega® applications indicate the possibility to rapidly and substantially increase EPA and to a smaller extent DHA concentrations in the epidermis of ex vivo human skin within a short period of time. This could likely be observed after including the emulsion in a pasty compound, in particular a cream, balm, gel, or ointment. A difference between the relative enrichment of EPA and DHA in skin samples reproduces several observations made by the inventors in various organs after Prontomega® IV injections in animals depleted of n-3 VLCFA. These studies had shown that the very low basal levels of EPA could be promptly and substantially raised, while the much higher basal concentrations of DHA led to much more limited enrichments; still, such enrichments on EPA and DHA concentrations led to substantial metabolic improvements (e.g., a reduction of liver steatosis and of insulin resistance). In addition, repeated applications to the skin samples and extended penetration period induced a further rise of the concentrations of EPA and DHA in the living epidermis. This opens the possibility to include emulsions with a high content (78-95%) of MCT together with an appropriate source of n-3 VLCFA into creams, balms, gels and/or ointments. For dermatologic and cosmetic preparations, the inventors favour a vegetable source such as oils derived from microalgae cultures over an animal source of n-3 VLCFA.

For applications on a human skin three applications should be done on the first day, with a two hours time period separating the applications. On the days following the first applications, two applications, for example one in the morning and one in the evening should take place. The treatment should be continued until improvement is noticed, e.g. a substantial reduction of inflammation and of the associated pain.

As described above, Prontomega® comprises fish oil as the sole source of n-3 VLCFA. However, molecules of n-3 VLCFA or alternative sources of n-3 VLCFA could complement or replace FO in the isotonic lipid emulsion and should be considered for the present invention. Substitutes or complements to FO could be, as the following examples:

an eicosapentaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as partial glycerides containing eicosapentaenoic acid, or as triglyceride comprising three eicosapentaenoic acid molecules; or

a docosapentaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as docosapentaenoic acid partial glyceride, or as triglyceride made of three docosapentaenoic acid molecules; or

a docosahexaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as docosahexaenoic acid partial glyceride, or as triglyceride made of three docosapentaenoic acid molecules.

Indeed, recent results suggest a more beneficial effect of EPA in protection cardiac ischemia while DHA offers an important protection after brain ischemia and spinal cord injury. DHA-rich preparations could also be administered pre-, per- and post-brain or spinal cord surgery.

It is also possible to use a combination of the eicosapentaenoic acid, and/or the docosapentaenoic acid, and/or the docosahexaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as partial glycerides, or as a combination of triglyceride molecules made of these omega-3 fatty acid molecules.

The present invention thus enables the medical staff to apply a method for the treatment of an acute heart ischemia which occurred on a day and caused an unstable angina and/or myocardial infarct. The method involves an intravenous bolus injection of an isotonic lipid emulsion which is repeated at time intervals during at least that day at which the heart ischemia occurred. The isotonic lipid emulsion to be injected comprises a 78 to 95% by weight of medium chain triglycerides and 5 to 22% by weight of a substance, that substance being either:

a fish oil, wherein the fish oil has an eicosapentaenoic acid content of from 20 to 50% by weight; or

an eicosapentaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as eicosapentaenoic acid partial glyceride, or as triglyceride comprising three eicosapentaenoic acid molecules; or

a docosapentaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as docosapentaenoic acid partial glyceride, or as triglyceride made of three docosapentaenoic acid molecules; or

a docosahexaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as docosahexaenoic acid partial glyceride, or as triglyceride made of three docosapentaenoic acid molecules; or

a combination of eicosapentaenoic acid, and/or docosapentaenoic acid, and/or docosahexaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as partial glycerides, or as a combination of triglyceride molecules made of these omega-3 fatty acid molecules.

A similar method can also be applied for a brain ischemia and/or other acute medical conditions cited in this description.

Many experiments described here before are focused on the treatment of an acute heart ischemia which occurred on a day and caused an unstable angina and/or myocardial infract, or on a brain ischemia. These experiments have proven that the injured area or volume of the body part affected by ischemia could be substantially reduced by repeated intravenous bolus injections of Prontomega®. The teaching that the injured area or volume of the heart or the brain can be reduced has opened the path to use repeated intravenous bolus injections of Prontomega® for the treatment or associated to the conventional treatment of the same or other diseases of the human or animal body. So, the isotonic lipid emulsion can be used in the treatment of an organ damage caused by a surgical operation, or after a traumatic injury (for example a brain injury or a spinal cord injury) which occurred on that human or animal body. For patients undergoing a surgical operation, the treatment can be applied either pre-, per- and post-operatively or post-trauma.

It is also possible to apply the treatment according to the invention for septic and/or metabolic complications occurring after a surgical operation or a trauma on a human or animal body. Further applications are on non-alcoholic fatty liver disease including non-alcoholic steato-hepatitis and hepatic complications of parenteral nutrition. Indeed, Prontomega® injections in rats have induced, within 2 h, a 40% reduction of liver triglycerides concentration (Carpentier Y A, Peltier S, Portois L, Sebedio J L, Leverve X, Malaisse W J, Rapid reduction of liver steatosis in omega3-depleted rats injected with a novel lipid emulsion. Horm Metab Res. 2008 December; 40(12):875-9. doi: 10.1055/s-0028-1083781. Epub 2008 Aug. 22. PMID: 18726832)

Where a transplantation of an organ of a human or animal body is required, the organ to be transplanted may be damaged by reactive oxygen and nitrogen species. In order to protect the organ to be transplanted against such damage it is proposed to apply to the donor patient an intravenous bolus injection of the isotonic lipid emulsion according to the invention. Such bolus injection is applied prior to the organ harvesting. In an analogous manner the isotonic lipid emulsion according to the invention can also be applied to the recipient patient for the preservation of a transplanted organ prior to and after grafting.

The invention also relates to a combined set of a preservation liquid mixture together with an isotonic lipid emulsion for use in the preservation of a to be transplanted organ of a human or animal body after harvesting and during shipping to a recipient patient. In such a manner the isotonic lipid emulsion according to the invention can be mixed to the preservation liquid mixture, which is generally used in the transport of organs to be transplanted.

The isotonic lipid emulsion according to the invention can also be used in the treatment of preterm and/or term neonates having an insufficient intake of docosahexaenoic acid. The treatment may be applied by repeated intravenous bolus injections of adapted volumes of the isotonic lipid emulsion.

The isotonic lipid emulsion according to the invention can further be used as a vehicle for carrying to a predetermined organ a predetermined drug and/or therapeutic or diagnostic agent. The treatment may be applied by repeated intravenous bolus injections of the isotonic lipid emulsion.

The treatment can also be used in acute and excessive inflammatory reactions or severe allergic reactions. Acute inflammation episodes are common in humans and animals and may respond to infectious or sterile stimuli. Such responses consist of an initial phase first induced by eicosanoids derived from n-6 arachidonic acid, followed by cytokines, chemokines, and complement components produced by immune cells. This phase is followed by the resolution phase. Many acute inflammatory responses are protective since they are self-limited and promptly followed by an active resolution phase. In less optimal conditions, the initial phase may be amplified or the resolution phase may be impaired. This may lead to an uncontrolled excessive reaction causing damage to the neighboring tissues and/or to chronic inflammation.

The initiation phase starts with migration of neutrophil (PMN) leukocytes out of capillary venules to tissues, upon attraction by chemotactic agents, namely eicosanoids derived from n-6 arachidonic acid. This is generally followed by PMN phagocytosis and neutralization of invaders.

The resolution phase starts with cessation of PMN influx and their apoptosis, followed by phagocytosis of various debris by macrophages, which is called efferocytosis. This step and the next ones are triggered by specialized pro-resolving mediators (SPMs) or resolvents which include lipoxins derived from the n-6 arachidonate, and several very potent mediators derived from EPA, DPA, and DHA, named the resolvins, protectins, and maresins. Each n-3 VLCFA is the precursor of one superfamily of mediators. In spite of differences in their structure, temporal code of biosynthesis and action, target organ and receptors, there is overlap and triggering action between different SPMs. They are all very potent and act at concentrations of nano- or picograms (Serhan C N and Levy B D: Resolvins in inflammation: emergence of the pro resolving superfamily of mediators, J Clin Invest, 2018, 128(7): 2657-2669).

SPMs inhibit translocation to the nucleus of NFkB transcription factor, which tightly regulates cytokine production via receptors expressed on innate lymphoid, NK, T, and B cells. This marked anti-inflammatory effect, which protects against cytokine storms (as recently reported in some SARS-CoV-2 patients), is also associated to a decrease of oxidative stress and a reduction of pain. Of importance, resolution of acute inflammation with SPMs does not impair immune defenses, which is in sharp contrast to common side effects of steroidal or non-steroidal anti-inflammatory drugs administration, with an exception for aspirin, which stimulates the biosynthesis of potent SPMs (Serhan C N et al: Resolvins, a family of bioactive products of omega-3 fatty acid transformation circuits initiated by aspirin treatment that counter pro-inflammation signals, J Exp Med, 2002; 196(8): 1025-1037). Indeed, SPMs do stimulate both cellular and humoral immunity. Such increase in host defenses reduces antibiotic requirement in septic conditions. In addition, while activated B cells increase immuglobulin IgM and IgG production and release, IgE production, namely occurring during allergic reactions, is reduced, which is particularly beneficial. Again, pain reduction is another important effect of SPMs during the resolution phase of inflammation.

Recent in vitro studies using cancer cells suggest a potential for resolvins to complement the administration of chemotherapeutic agents (Sulciner M L et al; Resolvins suppress tumor growth and enhance cancer therapy, J Exp Med, 2018, 215 (1) 115-140); indeed, apoptosis and necrosis of cancer cells lead to the formation of debris, which may stimulate growth of the surviving tumor cells. Since SPMs do enhance clearance of such cell debris by macrophages, they can be used as an adjuvant to optimize the efficacy of chemotherapeutic agents. MCT present in lipid formulations may enhance cell incorporation of SPMs, and their analogs, since they derive from n-3 VLCFA and have part of their molecule structure in common.

SPMs are also produced in the brain and the spinal cord where they exert substantial neuroprotective activities, e.g. against microglial inflammation and after damage caused by surgical, traumatic, or ischemic injuries (Nielsen M M et al, Mass spectrometry imaging of biomarker lipids for phagocytosis and signaling during focal cerebral ischemia, Sci Rep 2016; 6:39571).

SPMs are namely present in tissues, such as lymph nodes, spleen, serum, breast milk, placenta, but also in tears and inflammatory exudates. Many SPMs do not reach the circulation but remain in tissues where they are inactivated at local sites of inflammation, in particular by an eicosanoid oxidoreductase.

Some resolvins, e.g., resolvin E1 or RvE1, have been applied with success in clinical conditions such as dry eye disease and periodontal inflammation. Potent mimetics and stable analogs have been synthetized which are resistant to neutralization by oxidoreductase and provide a longer duration of activity.

Since SPMs have part of their molecule in common with their n-3 VLCFA precursors, it is proposed to include some SPMs and/or stable analogs to be mixed in IV lipid emulsions with a high proportion of MCT according to the invention, for promptly treating overly exuberant and uncontrolled inflammatory reactions, in particular those caused by severe, surgical or traumatic, injuries, or by septic episodes, for example of bacterial, viral, or fungal origin, or by allergic reactions.

Based on the rapid incorporation of EPA and DHA in the epidermis (see supra pp 32-35 after application of Prontomega®, other preparations are considered for treating inflammatory or allergic diseases of the skin, or for skin burns prevention and treatment, by topical applications of emulsions made of a high proportion of MCT together with oils preferably derived from cultures of selected microalgae and selectively enriched in either EPA, or DPA, or DHA, or selected mixtures of n-3 VLCFA. These emulsions can be used for skin application, for example in the form of creams, balms, gels, and/or ointments, or other pasty compounds. Furthermore, these emulsions can be used in cosmetics aimed at providing different protective effects, e.g. against damage caused by exposition to UV, or against skin ageing or even for purely cosmetic effects.

In particular a pasty compound, such as a cream, balm, gel, or ointment comprising an isotonic nano particle lipid emulsion according to the invention can be used in the skin protection against or treatment of burns, in particular skin burns, sunburns, or burns by laser or radiation therapies, where treatment is applied by skin applications, the isotonic lipid emulsion comprising a 78 to 95% by weight of medium chain triglycerides and 5 to 22% by weight of algal oil, wherein the algal oil has a concentration of from 0 to 99.5% of eicosapentaenoic acid, or of docosapentaenoic acid, or of docosahexaenoic acid, or a mixture of these n-3 VLCFA.

The pasty compound can also be used to reduce the inflammatory reaction following skin laser therapy, or in the skin treatment of severe cutaneous allergic reactions as well as insect bites, all conditions where treatment is applied by dermal applications. Furthermore the isotonic lipid emulsion can be for used in the treatment of skin diseases of a human or animal, where the treatment is applied by skin applications, or for use in the treatment of an organ disease of a human or animal body, where treatment of the organ disease is realised by skin application.

The emulsions according to the invention may even be used as a component of eye droplets or creams and used to treat ocular allergic and other inflammatory conditions, such as for example conjunctivitis, uveitis, and blepharitis. Up to four applications per day could be considered.

To briefly recapitulate, n-3 VLCFA are essential for health, but their intake is declining in most regions of the world. They act, either directly (by affecting the physical properties of cell membranes and reducing their raft content), or after conversion into potent derivatives (eicosanoids and/or SPMs), largely by regulating the expression of several genes.

Recognized properties of n-3 VLCFA include: anti-inflammatory and anti-oxidant effects; maintenance of cell mitochondrial function, protection of endothelial function resulting in improved tissue perfusion, blood pressure lowering, decreased cardiac rhythm, enhanced wound healing, preservation of muscle mass and improved glucose and lipid homeostasis, etc. . . . . These effects may be particularly relevant in acute conditions resulting from different etiologies, but they require a prompt incorporation of n-3 VLCFA or their derivatives in cell membranes.

The present invention offers solutions to fulfill this pre-requisite by intravenous injections repeated at short-term intervals of lipid emulsions containing a high proportion of MCT and n-3 VLCFA or selected derivatives. Unexpectedly, the inventors observed that MCT may not only beneficially affect the blood clearance of n-3 containing emulsions (allowing for the use via repeated bolus injections), but also that they markedly facilitate the incorporation of n-3 VLCFA in cell membranes. The inventors also propose to use selected preparations for skin or ocular applications.

1. Isotonic lipid emulsion for use in the treatment of an acute heart ischemia causing an unstable angina and/or myocardial infarct, where the treatment is applied by intravenous bolus injection which is repeated at time intervals during at least a day at which the heart ischemia occurred, the isotonic lipid emulsion comprising a 78 to 95% by weight of medium chain triglycerides and 5 to 22% by weight of a substance, said substance being either:
a fish oil, wherein the fish oil has an eicosapentaenoic acid content of from 20 to 50% by weight; or
an eicosapentaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as eicosapentaenoic acid partial glyceride, or as triglyceride comprising three eicosapentaenoic acid molecules; or
a docosapentaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as docosapentaenoic acid partial glyceride, or as triglyceride made of three docosapentaenoic acid molecules; or
a docosahexaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as docosahexaenoic acid partial glyceride, or as triglyceride made of three docosapentaenoic acid molecules; or
a combination of eicosapentaenoic acid, and/or docosapentaenoic acid, and/or docosahexaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as partial glycerides, or as a combination of triglyceride molecules made of these omega-3 fatty acid molecules.

2. Isotonic lipid emulsion for use in the treatment of a brain ischemia causing a stroke, where the treatment is applied by intravenous bolus injection which is repeated at time intervals during at least a day at which the brain ischemia occurred, the isotonic lipid emulsion comprising a 78 to 95% by weight of medium chain triglycerides and 5 to 22% by weight of a substance, said substance being either:
a fish oil, wherein the fish oil has a docosahexaenoic acid content of from 20 to 50% by weight; or
a docosahexaenoic acid, either as pure omega-3 fatty acid molecules, or as ethyl ester derivative, or as docosahexaenoic acid partial glyceride, or as triglyceride made of three docosahexaenoic acid molecules; or
an eicosapentaenoic acid, either as pure omega-3 fatty acid molecules, or as ethyl ester or phospholipid esters, in particular phosphatidylserine (PS), phosphatidylcholine (PC), or phosphatidylethanolamine (PE) derivatives, or as eicosapentaenoic acid partial glyceride, or as triglyceride made of three eicosapentaenoic acid molecules; or
a docosapentaenoic acid, either as pure omega-3 fatty acid molecules, or as ethyl ester derivatives, or as docosapentaenoic acid partial glyceride, or as triglyceride made of three docosapentaenoic acid molecules; or
a combination of eicosapentaenoic, and/or docosapentaenoic acid, and/or docosahexaenoic acid, either as pure omega-3 fatty acid molecules, or as ethyl ester or phospholipid esters, in particular phosphatidylserine (PS), phosphatidylcholine (PC), or phosphatidylethanolamine (PE) derivatives, or as partial glycerides, or as a combination of triglyceride molecules made of these omega-3 fatty acids.

3. Isotonic lipid emulsion for use in the treatment of an organ damage of a human or animal body caused by a surgical operation or after a traumatic injury which occurred on that body, where the treatment is applied, in particular either pre-, per and postoperatively or post—the traumatic injury, by intravenous bolus injection which is repeated at time intervals during at least a day at which the organ damage occurred, the isotonic lipid emulsion comprising a 78 to 95% by weight of medium chain triglycerides and 5 to 22% by weight of a substance, said substance being either:
a fish oil, wherein the fish oil has an eicosapentaenoic acid content of from 20 to 50% by weight; or
an eicosapentaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as eicosapentaenoic acid partial glyceride, or as triglyceride made of three eicosapentaenoic acid molecules; or
a docosapentaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as docosapentaenoic acid partial glyceride, or as triglyceride made of three docosapentaenoic acid molecules; or
a docosahexaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as docosahexaenoic acid partial glyceride, or as triglyceride made of three docosapentaenoic acid molecules; or
a combination of eicosapentaenoic acid, and/or docosapentaenoic acid, and/or docosahexaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as partial glycerides, or as a combination of triglyceride molecules made of these omega-3 fatty acids.

4. Isotonic lipid emulsion for use in the treatment of a brain injury caused either by a surgical operation or by trauma, where the treatment is applied by intravenous bolus injection, either pre-, per- and post-operatively or post-trauma, which treatment is repeated at time intervals during at least within a day at which the brain injury occurred, the isotonic lipid emulsion comprising a 78 to 95% by weight of medium chain triglycerides and 5 to 22% by weight of a substance, said substance being either:
a fish oil, wherein the fish oil has a docosahexaenoic acid content of from 20 to 50% by weight; or
a docosahexaenoic acid, either as pure omega-3 fatty acid molecules, or as ethyl ester or phospholipid esters, in particular phosphatidylserine (PS), or phosphatidylcholine (PC) phosphatidylethanolamine (PE) derivative, or as docosahexaenoic acid partial glyceride, or as triglyceride made of three docosahexaenoic acid molecules; or
an eicosapentaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as eicosapentaenoic acid partial glyceride, or as triglyceride made of three eicosapentaenoic acid molecules; or
a docosapentaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as docosapentaenoic acid partial glyceride, or as triglyceride made of three docosapentaenoic acid molecules; or
a combination of eicosapentaenoic, and/or docosapentaenoic acid, and/or docosahexaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as partial glycerides, or as a combination of triglyceride molecules made of these omega-3 fatty acids.

5. Isotonic lipid emulsion for use in the treatment of a spinal cord injury, in particular caused either by a surgical operation or a trauma, which treatment is applied, either pre-, per- and post-operatively or post-trauma, by intravenous bolus injection which is repeated at time intervals during at least a day at which the spinal cord injury occurred, the isotonic lipid emulsion comprising a 78 to 95% by weight of medium chain triglycerides and 5 to 22% by weight of a substance, said substance being either;
a fish oil, wherein the fish oil has a docosahexaenoic acid content of from 20 to 50% by weight; or
a docosahexaenoic acid, either as pure omega-3 fatty acid molecules, or as ethyl ester or phospholipid esters, in particular phosphatidylserine (PS), phosphatidylcholine (PC) or phosphatidylethanolamine (PE) derivative, or as docosahexaenoic acid partial glyceride, or as triglyceride made of three docosahexaenoic acid molecules; or
an eicosapentaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as eicosapentaenoic acid partial glyceride, or as triglyceride made of three eicosapentaenoic acid molecules; or
a docosapentaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as docosapentaenoic acid partial glyceride, or as triglyceride made of three docosapentaenoic acid molecules; or
a combination of eicosapentaenoic, and/or docosapentaenoic acid, and/or docosahexaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as partial glycerides, or as a combination of triglyceride molecules made of these omega-3 fatty acids.

6. Isotonic lipid emulsion for use in the treatment of acute and excessive inflammatory reactions or severe allergic reactions, where the treatment is applied by intravenous bolus injection which is repeated at time intervals during at least a day at which the inflammatory reactions occurred, the isotonic lipid emulsion comprising a 78 to 95% by weight of medium chain triglycerides and 5 to 22% by weight of a substance, said substance being either;
a fish oil, wherein the fish oil has an eicosapentaenoic acid content of from 20 to 50% by weight; or
an eicosapentaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as eicosapentaenoic acid partial glyceride, or as triglyceride made of three eicosapentaenoic acid molecules; or
a docosahexaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as docosahexaenoic acid partial glyceride, or as triglyceride made of three docosahexaenoic acid molecules; or
a docosapentaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as docosapentaenoic acid partial glyceride, or as triglyceride made of three docosapentaenoic acid molecules; or
a combination of eicosapentaenoic, and/or docosapentaenoic acid, and/or docosahexaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as partial glycerides, or as a combination of triglyceride molecules made of these omega-3 fatty acids.

7. Isotonic lipid emulsion for use in the treatment of septic and/or metabolic complications occurring after a surgical operation or a trauma on a human or animal body, where the treatment is applied by intravenous bolus injection which is repeated at time intervals during at least a day at which the treatment occurred, the isotonic lipid emulsion comprising a 78 to 95% by weight of medium chain triglycerides and 5 to 22% by weight of a substance, said substance being either;
a fish oil, wherein the fish oil has an eicosapentaenoic acid content of from 20 to 50% by weight; or
an eicosapentaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as eicosapentaenoic acid partial glyceride, or as triglyceride made of three eicosapentaenoic acid molecules; or
a docosahexaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as docosahexaenoic acid partial glyceride, or as triglyceride made of three docosahexaenoic acid molecules; or
a docosapentaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as docosapentaenoic acid partial glyceride, or as triglyceride made of three docosapentaenoic acid molecules; or
a combination of eicosapentaenoic, and/or docosapentaenoic acid, and/or docosahexaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as partial glycerides, or as a combination of triglyceride molecules made of these omega-3 fatty acids.

8. Isotonic lipid emulsion for use in the treatment of a non-alcoholic fatty liver disease including non-alcoholic steato-hepatitis and hepatic complications of parenteral nutrition, where the treatment is applied by intravenous bolus injection which is repeated at time intervals during at least a day of the treatment and preferably on the subsequent days after said treatment started, the isotonic lipid emulsion comprising a 78 to 95% by weight of medium chain triglycerides and 5 to 22% by weight of a substance, said substance being:
a fish oil, wherein the fish oil has an eicosapentaenoic acid content of from 20 to 50% by weight; or
an eicosapentaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as eicosapentaenoic acid partial glyceride, or as triglyceride made of three eicosapentaenoic acid molecules; or
a docosahexaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as docosahexaenoic acid partial glyceride, or as triglyceride made of three docosahexaenoic acid molecules; or
a docosapentaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as docosapentaenoic acid partial glyceride, or as triglyceride made of three docosapentaenoic acid molecules; or
a combination of eicosapentaenoic, and/or docosapentaenoic acid, and/or docosahexaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as partial glycerides, or as a combination of triglyceride molecules made of these omega-3 fatty acids.

9. Isotonic lipid emulsion for use in the protection of a to be transplanted organ of a human or animal body against damage by reactive oxygen and nitrogen species, where the treatment is applied to a donor patient prior to and eventually until completion of organ harvesting by intravenous bolus injection the isotonic lipid emulsion comprising a 78 to 95% by weight of medium chain triglycerides and 5 to 22% by weight of a substance, said substance being either:
a fish oil, wherein the fish oil has an eicosapentaenoic acid content of from 20 to 50% by weight and a docosahexaenoic content of from 20 to 50% by weight; or
an eicosapentaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as eicosapentaenoic acid partial glyceride, or as triglyceride made of three eicosapentaenoic acid molecules; or
a docosapentaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as docosapentaenoic acid partial glyceride, or as triglyceride made of three docosapentaenoic acid molecules; or
a docosahexaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as docosahexaenoic acid partial glyceride, or as triglyceride made of three docosahexaenoic acid molecules; or
a combination of eicosapentaenoic, and/or docosapentaenoic acid, and/or docosahexaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as partial glycerides, or as a combination of triglyceride molecules made of these omega-3 fatty acids.

10. A combined set of a preservation liquid mixture and an isotonic lipid emulsion for use in the preservation of a to be transplanted organ of a human or animal body against damage by reactive oxygen and nitrogen species after harvesting and during shipping to a recipient patient, the isotonic lipid emulsion comprising a 78 to 95% by weight of medium chain triglycerides and 5 to 22% by weight of a substance, said substance being either:
a fish oil, wherein the fish oil has an eicosapentaenoic acid content of from 20 to 50% by weight and a docosahexaenoic content of from 20 to 50% by weight; or
an eicosapentaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as eicosapentaenoic acid partial glyceride, or as triglyceride made of three eicosapentaenoic acid molecules; or
a docosapentaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as docosapentaenoic acid partial glyceride, or as triglyceride made of three docosapentaenoic acid molecules; or
a docosahexaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as docosahexaenoic acid partial glyceride, or as triglyceride made of three docosahexaenoic acid molecules; or
a combination of eicosapentaenoic, and/or docosapentaenoic acid, and/or docosahexaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as partial glycerides, or as a combination of triglyceride molecules made of these omega-3 fatty acids.

11. Isotonic lipid emulsion for use in the preservation of a transplanted organ of a human or animal body against damage by reactive oxygen and nitrogen species prior to and after grafting to a recipient patient, the isotonic lipid emulsion comprising a 78 to 95% by weight of medium chain triglycerides and 5 to 22% by weight of a substance, said substance being either;
a fish oil, wherein the fish oil has an eicosapentaenoic acid content of from 20 to 50% by weight and a docosahexaenoic content of from 20 to 50% by weight; or
an eicosapentaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as eicosapentaenoic acid partial glyceride, or as triglyceride made of three eicosapentaenoic acid molecules; or
a docosapentaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as docosapentaenoic acid partial glyceride, or as triglyceride made of three docosapentaenoic acid molecules; or
a docosahexaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as docosahexaenoic acid partial glyceride, or as triglyceride made of three docosahexaenoic acid molecules; or
a combination of eicosapentaenoic, and/or docosapentaenoic acid, and/or docosahexaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as partial glycerides, or as a combination of triglyceride molecules made of these omega-3 fatty acids.

12. Isotonic lipid emulsion for use in the treatment of preterm and/or term neonates having an insufficient intake of docosahexaenoic, where the treatment is applied by intravenous bolus injection, the isotonic lipid emulsion comprising a 78 to 95% by weight of medium chain triglycerides and 5 to 22% by weight of a substance, said substance being either:
a fish oil, wherein the fish oil has a docosahexaenoic acid content of from 20 to 50% by weight; or
a docosahexaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as docosahexaenoic acid partial glyceride, or as triglyceride made of three docosapentaenoic acid molecules.

13. Isotonic lipid emulsion for use in the treatment of infants or children requiring parenteral nutrition, in prevention and/or treatment of parenteral nutrition associated liver dysfunction, where the treatment is applied by intravenous bolus injection which is repeated at time intervals during at least a day of the treatment, the isotonic lipid emulsion comprising a 78 to 95% by weight of medium chain triglycerides and 5 to 22% by weight of a substance, said substance being either:
a fish oil, wherein the fish oil has a docosahexaenoic acid content of from 20 to 50% by weight; or
an eicosapentaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as eicosapentaenoic acid partial glyceride, or as triglyceride made of three eicosapentaenoic acid molecules; or
a docosapentaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as docosapentaenoic acid partial glyceride, or as triglyceride made of three docosapentaenoic acid molecules; or
a docosahexaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as docosahexaenoic acid partial glyceride, or as triglyceride made of three docosapentaenoic acid molecules; or
a combination of eicosapentaenoic, and/or docosapentaenoic acid, and/or docosahexaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as partial glycerides, or as a combination of triglyceride molecules made of these omega-3 fatty acids.

14. Isotonic lipid emulsion as claimed in anyone of the claims 1 to 13, wherein the treatment is repeated over subsequent days following said intravenous bolus injection.

15. Isotonic lipid emulsion for use as vehicle for carrying to a predetermined organ a predetermined drug and/or therapeutic agent, where treatment is applied by intravenous bolus injection, possibly repeated at time intervals during the day of the treatment, the isotonic lipid emulsion comprising a 78 to 95% by weight of medium chain triglycerides and 5 to 22% by weight of fish oil, wherein the fish oil has an eicosapentaenoic acid and docosahexaenoic acid content of from 20 to 40% based on the fatty acid methyl esters of the fish oil.

16. Isotonic lipid emulsion for use as vehicle for carrying to a predetermined organ a predetermined diagnostic agent, where use is applied by intravenous bolus injection, the isotonic lipid emulsion comprising a 78 to 95% by weight of medium chain triglycerides and 5 to 22% by weight of fish oil, wherein the fish oil has an eicosapentaenoic acid and docosahexaenoic acid content of from 20 to 40% based on the fatty acid methyl esters of the fish oil.

17. A pasty compound, in particular a cream, balm, gel, or ointment comprising an isotonic nano particle lipid emulsion for use in the treatment of skin diseases of a human or animal, where the treatment is applied by skin applications, the isotonic nano particle lipid emulsion comprising a 78 to 95% by weight of medium chain triglycerides and 5 to 22% by weight of algal oil, wherein the algal oil has either an:
eicosapentaenoic acid of from 0 to 99.5% by weight based on the fatty acid methyl esters of the algal oil; or
a docosapentaenoic acid of from 0 to 99.5% based on the fatty acid methyl esters of the algal oil; or
a docosahexaenoic acid of from 0 to 99.5% based on the fatty acid methyl esters of the algal oil; or
a combination of a eicosapentaenoic acid and a docosapentaenoic acid and a docosahexaenoic acid of from 0 to 99.5% based on the fatty acid methyl esters of the algal oil.

18. A pasty compound, in particular a cream, balm, gel, or ointment comprising an isotonic nano particle lipid emulsion for use in the treatment of an organ disease of a human or animal body, where treatment of the organ disease is realised by skin application, the isotonic nano particle lipid emulsion comprising a 78 to 95% by weight of medium chain triglycerides and 5 to 22% by weight of a combination of omega-3 very long-chain fatty acids produced from cultured microalgae, wherein the combination has an eicosapentaenoic acid content of from 0 to 99.50%, a docosapentaenoic content of 0 to 99.50%, and a docosahexaenoic content of 0 to 99.50%, based on the fatty acid methyl esters of the algal oil.

19. A pasty compound, in particular a cream, balm, gel, or ointment comprising an isotonic nano particle lipid emulsion for use in the skin protection against or treatment of burns, in particular skin burns, sunburns, or burns by laser or radiation therapies, where treatment is applied by skin applications, the isotonic lipid emulsion comprising a 78 to 95% by weight of medium chain triglycerides and 5 to 22% by weight of algal oil, wherein the algal oil has a docosapentaenoic acid of from 0 to 99.5% based on the fatty acid methyl esters of the algal oil.

20. A pasty compound, in particular a cream, balm, gel, or ointment comprising an isotonic nano particle lipid emulsion for use in the skin treatment of severe cutaneous allergic reactions as well as insect bites, where treatment is applied by skin applications, the isotonic lipid emulsion comprising a 78 to 95% by weight of medium chain triglycerides and 5 to 22% by weight of algal oil, wherein the algal oil has an eicosapentaenoic acid of from 0 to 99.5%, a docosapentaenoic content of 0 to 99.5%, and a docosahexaenoic content of 0 to 99.5%, based on the fatty acid methyl esters of the algal oil.

21. A pasty compound, in particular a cream, balm, gel, or ointment comprising an isotonic nano particle lipid emulsion for use as a skin cosmetic, the isotonic lipid emulsion comprising a 78 to 95% by weight of medium chain triglycerides and 5 to 22% by weight of microalgal oil, wherein the algal oil has an eicosapentaenoic acid of from 0 to 99.5%, a docosapentaenoic content of 0 to 99.5%, and a docosahexaenoic content of 0 to 99.5%, based on the fatty acid methyl esters of the algal oil.

22. An isotonic lipid emulsion comprising a 78 to 95% by weight of medium chain triglycerides and 5 to 22% by weight of a substance, and further comprising selected specialized pro-resolving mediators, in particular neuroprotectin D1 and resolvins D1, D2, and D4, and maresins 1 and 2, and more particularly neuroprotectin D1 and resolvin D1, said substance being either:
a fish oil, wherein the fish oil has an eicosapentaenoic acid content of from 20 to 50% by weight; or
an eicosapentaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as eicosapentaenoic acid partial glyceride, or as triglyceride comprising three eicosapentaenoic acid molecules; or
a docosapentaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as docosapentaenoic acid partial glyceride, or as triglyceride made of three docosapentaenoic acid molecules; or
a docosahexaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as docosahexaenoic acid partial glyceride, or as triglyceride made of three docosapentaenoic acid molecules; or
a combination of eicosapentaenoic acid, and/or docosapentaenoic acid, and/or docosahexaenoic acid, either as pure omega-3 fatty acid molecules, or as ethyl ester or phospholipid esters, in particular phosphatidylcholine (PC), phosphatidylserine (PS) or phosphatidylethanolamine (PE) derivatives, or as partial glycerides, or as a combination of triglyceride molecules made of these omega-3 fatty acid molecules.

23. An eye droplet composition comprising an isotonic nano particle lipid emulsion for use in the treatment of an eye disease of a human or animal, the isotonic nano particle lipid emulsion comprising a 78 to 95% by weight of medium chain triglycerides and 5 to 22% by weight of algal oil, wherein the algal oil has either an:
eicosapentaenoic acid of from 0 to 99.5% by weight based on the fatty acid methyl esters of the algal oil; or
a docosapentaenoic acid of from 0 to 99.5% based on the fatty acid methyl esters of the algal oil; or
a docosahexaenoic acid of from 0 to 99.5% based on the fatty acid methyl esters of the algal oil; or
a combination of a eicosapentaenoic acid and a docosapentaenoic acid and a docosahexaenoic acid of from 0 to 99.5% based on the fatty acid methyl esters of the algal oil.
